(19) United States
(12) Patent Application Publication
(10) Pub. No.: US 2025/0043270 A1
(43) Pub. Date: Feb. 6, 2025

(54) A METHOD OF TREATING ACUTE MYELOID LEUKEMIA

(71) Applicant: Agency for Science, Technology and Research, Singapore (SG)

(72) Inventors: Amit Singhal, Singapore (SG); Kumar Dilip, Singapore (SG)

(21) Appl. No.: 18/715,366

(22) PCT Filed: Nov. 30, 2022
(86) PCT No.: PCT/SG2022/050872

§ 371 Date: May 31, 2024

(30) Foreign Application Priority Data
Dec. 1, 2021 (SG) 10202113390W

(51) Int. Cl. C12N 15/10; A61K 35/28; A61K 45/06; A61P 35/02; C12N 5/0789

(52) U.S. Cl. CPC C12N 15/102; A61K 35/28; A61K 45/06; A61P 35/02; C12N 5/0647

(57) ABSTRACT

There is provided a genetically modified cell wherein at least one gene has been deleted from the cell and the gene is selected from the group consisting of p50, p52, p65, c-Rel, and RelB. Also disclosed are methods of identifying a target for treating acute myeloid leukemia (AML) in a subject, methods of treating acute myeloid leukemia (AML) in a subject in need thereof, comprising inhibiting the activity of an NF-KB pathway. Also provided is an ATP13A2 inhibiting agent for use in therapy or medicine, for use in treating AML, and in the manufacture of a medicament for treating AML.

### TECHNICAL FIELD

The present disclosure relates broadly to a method of identifying a therapeutic target for treating acute myeloid leukemia (AML) using a genetically modified cell and a method of treating AML.

### BACKGROUND

Acute myeloid leukemia (AML) is a cancer of the myeloid line of blood cells, interfering with normal blood cell production due to the rapid growth of abnormal cells in the bone marrow and blood. AML progresses rapidly and if left untreated is typically fatal within weeks or month. AML affects about one million people in 2015 with 147,000 deaths globally.

The remission-inducing chemotherapy with cytarabine and anthracycline have been the mainstay of AML therapy for the past four decades, achieving complete remission (CR) in 60-80% of patients <60 years of age. However, the 5-year overall survival (OS) among AML patients remains a challenge due to the relapse which led to an intense effort to discover promising targeted therapies. Despite the rapid advancement in the therapeutic approaches for acute myeloid leukaemia (AML), primary and secondary drug resistance to the current therapies remains a pervasive issue and thus the need to identify and develop the next class of AML drugs, can lead to the design of novel combination regimens to increase the cure rates in AML, to prolong remission duration and survival, and improve quality of life of AML patients.

Therefore, there is a need to provide an alternative method to treat AML.

### SUMMARY

In one aspect, there is provided a genetically modified cell wherein at least one gene has been deleted from the cell and the gene is selected from the group consisting of p65 (RELA), p50 (NFKB1), p52 (NFKB2), c-Rel (REL) and RelB (RELB).

In another aspect, there is provided a method of identifying a therapeutic target for treating acute myeloid leukemia (AML) in a subject, the method comprising using the cell as described herein.

In yet another aspect, there is provided a method of treating acute myeloid leukemia (AML) in a subject in need thereof, comprising modulating the activity of an NF-KB pathway.

In some examples, the perturbation of an NF-KB pathway modulates multiple pathways comprising a metabolic pathway, an inflammatory pathway, a cancer associated pathway, and combinations thereof.

In some examples, the NF-KB pathway gene is p65.

In some examples, the metabolic pathway comprises oxidative phosphorylation (OXPHOS), mitochondrial dysfunction, Sirtuin signaling and/or mTOR signaling.

In some examples, the cancer associated pathway comprises pathways related to acute myeloid leukemia (AML), small lung cancer, and/or pancreatic adenocarcinoma, optionally, wherein the cancer associated pathway comprises AML and AML-associated signaling pathways comprising IL-6, IL-7, JAK, CXCR4, JAK-STAT and/or GM-CSF.

In some examples, the inflammatory pathway comprises TLR, TNFR2, CCL22, IL-10, IL-17A, CD40 and/or IL-6 signalling.

In some examples, the NF-KB pathway regulates gene and/or protein expression of a lysosomal-associated protein.

In some examples, the NF-KB pathway regulates the function of a lysosomal-associated protein.

In some examples, the gene of lysosomal-associated protein is ATP13A2. The respective protein is known as Park9.

In some examples, the method comprises administering an agent that inhibits the activity of the ATP13A2 gene.

In some examples, the treatment reduces one or more indications comprising reduction of oxygen consumption rate (OCR), reduction in mitochondrial maximum respiration, reduction in mitochondrial spare respiratory capacity, reduction of nonmitochondrial respiration, and combinations thereof.

In some examples, the treatment reduces one or more indications comprising reduction in glycolysis, reduction in glycolytic capacity, and combinations thereof.

In some examples, the treatment reverses or improves dysfunctional mitochondrial function.

In some examples, the treatment reduces one or more indications comprising elimination of ability of leukemia stem cells (LSC) to proliferate and/or form colonies.

In some examples, the subject is further treated with inhibiting agents comprising a chemotherapeutic agent, an immune therapy agent, a cellular therapy agent, an oligonucleotide, an antigen binding molecule, a small molecule inhibitor, and/or combinations thereof.

In some examples, the subject is further treated with a chemotherapeutic agent.

In yet another aspect, there is provided an ATP13A2 inhibiting agent for use in therapy or medicine.

In yet another aspect, there is provided an ATP13A2 inhibiting agent for use in treating AML.

In yet another aspect, there is provided the use of an ATP13A2 inhibiting agent in the manufacture of a medicament for treating AML.

### Definitions

The term “treating”, “treat” and “therapy”, and synonyms thereof refer to both therapeutic treatment and prophylactic or preventative measures, wherein the object is to prevent or slow down (lessen) a medical condition, which includes but is not limited to diseases (such as acute myeloid leukemia/AML), symptoms and disorders. A medical condition also includes a body's response to a disease or disorder, e.g., dysregulated cell proliferation, dysregulated cell metabolism, and/or inflammation. Those in need of such treatment include those already with a medical condition as well as those prone to getting the medical condition or those in whom a medical condition is to be prevented.

The term “subject” as used herein includes patients and non-patients. The term “patient” refers to individuals suffering or are likely to suffer from a medical condition such as an acute myeloid leukemia/AML, while “non-patients” refer to individuals not suffering and are likely to not suffer from the medical condition. “Non-patients” include healthy individuals, non-diseased individuals and/or an individual free from the medical condition. The term “subject” includes humans and animals. Animals may include, but is not limited to, mammals (for example non-human primates, canine, murine and the like), and the like. “Murine” refers to any mammal from the family Muridae, such as mouse, rat, rabbit, and the like. The term “micro” as used herein is to be interpreted broadly to include dimensions from about 1 micron to about 1000 microns.

The term “reverses” as used herein refers to reducing the symptoms stemming from a disease, which includes undoing the damage that the disease has caused and truly heal the body or restoring a key cellular function that was blocked or affected such as by a disease.

The term “improves” as used herein refers to the act or process of making better either symptoms or prognosis of a disease or key cellular function.

The term “nano” as used herein is to be interpreted broadly to include dimensions less than about 1000 nm.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other, or element A may contain element B or vice versa.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals. Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc.,) spanning the ranges. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

The NF-kB family of transcription factors (TFs) are represented by five canonical and noncanonical members: p65 (RELA), RelB (RELB), c-Rel (REL), p50/p105 (NF-κB1) and p52/p100 (NF-κB2). These TFs share a common Rel homology domain that mediates their dimerization and their subsequent binding to functional DNA promoter elements which results in the downstream modulation of gene expression in a tissue and context dependent manner. NF-kB TFs binds to genomic regions in a homo- or heterodimeric fashion and regulates diverse biological functions including development, hematopoiesis, metabolism, immune function, etc., in a tightly regulated fashion.

NF-kB pathways are frequently deregulated in most hematological malignancies and could be directly driven by genetic mutations within associated regulatory genes such as in lymphomas and multiple myeloma (MM). These mutations can also drive acute myeloid leukemia (AML), a genetically heterogeneous group of clonal stem cell malignancies arising from leukemic stem cells (LSC), pathogenesis. In fact, constitutive NF-kB pathways activation in 40% of AML patients has been reported but is shown to be indirectly driven by AML associated genetic mutations. One of the frequently reported genetic drivers of AML is PML-RARA (Promyelocytic Leukemia—Retinoic Acid Receptor Alpha) gene fusion, which impairs myeloid differentiation and also inhibits NF-kB pathway through p65. NF-kB TFs, and specifically p65, have also been linked to metabolic reprograming and adaptation in cancers. Furthermore, cytokines positive feedback loop and increased levels of active proteasome machinery also plays role in the constitutive NF-kB activity.

The last decade has seen expanded investigations focused on understanding the deregulation of NF-kB pathways in hematological malignancies, which have led to the search for the clinical implication of NF-kB inhibitors which mostly act indirectly. Apart from promising outcomes of proteasome inhibitor in MM which indeed is linked to activation of canonical NF-kB pathway, efforts to target this biology in other hematological malignancies have not been very successful. This has raised concerns about the general strategy of a global targeting of NF-kB pathways and sought for a factor and context specific strategy to address issues associated with NF-kB pathway inhibition in clinical settings. Much of the current understanding of the role of individual NF-kB family TFs in AML pathogenesis are based on findings from biochemical assays, in vitro modelling, and animal studies and human specific pathophysiological functions are remain poorly understood at the molecular level. This necessitates a more systematic study of this biology to identify the specific roles of individual NF-kB TFs in AML pathogenesis.

Opposing roles of NF-kB in cancers challenges the general assumption of prooncogenic property, however human specific pathophysiological functions of NF-kB signaling pathways remains poorly understood. The inventors of the present disclosure utilized the deregulated NF-kB signaling pathway in AML as a probe to identify and characterize novel therapeutic targets. The inventors of the present disclosure have systematically investigated the pathophysiological functions of the individual NF-kB TFs in human AML cellular model (i.e U937) by utilizing a novel oligomer based dual gRNA CRISPR-Cas9 mediated knockout (KO) approach, where the inventors of the present disclosure generated U937 cells devoid of individual NF-kB TF: p50, p52, p65, c-Rel and RelB. Integration of global differential gene expression (DEG) data from the five NF-kB TFs KO cells identified a TF specific gene signature. This TF-specific signature when combined with the published NF-kB TFs chromatin immunoprecipitation (ChIP) data, identified the deregulation in cancer and metabolic pathway genes among p65 DEGs. Using metabolic and leukemic stem cell (LSCs) proliferation analysis, and AML xenograft studies in humanized mice (hu-NOG), the inventors of the present disclosure showed that p65 deficient AML cells display (i) deregulation in cellular bioenergetics, (ii) increased LSCs properties, (iii) enhanced pathogenesis in humanized NOG mice xenografts. Categorization of AML patients using p65^{-KO}-specific gene signature identified ATP13A2, a lysosomal transporter, as a predictive marker of the patient's therapy response and overall survival (OS). Impaired expression/inhibition of ATP13A2 in p65 KO reversed, (i) cellular bio-energetic state and linked lysosomal deregulation, (ii) LSCs properties (iii) pathogenesis in p65-KO induced xenografts. The results of the present disclosure support an anti-leukemic function of p65, linked to metabolic plasticity, and unravel an important role of ATP13A2 in human-related AML pathology. Furthermore, the inventors showed a p65-ATP13A2 axis mediated cellular energetic adaptation, LSCs functions which ultimately translated to aggressive AML pathologies.

Constitutive NF-kB activity observed in AML patients has propelled NF-kB as an attractive target for AML. However, clinical trials targeting NF-kB has not produced encouraging clinical outcomes. The study of the present disclosure suggests p65-dependent stratification and provide ATP13A2 as a novel therapeutic target against AML. The present invention relates to the identification of novel therapeutic targets of AML, deciphered using gene signatures from pathologically aggressive NFκB-p65 deficient AML cell line. The top candidate gene of the gene signature, ATP13A2, when knocked-down resulted in the reversal of cellular energetic adaptation and aggressive phenotype of p65 deficient AML cells in novel humanized-mice xenograft model. The findings of the present disclosure suggest ATP13A2 as a novel therapeutic target against AML.

In one aspect, there is provided a genetically modified cell wherein at least one gene has been deleted (or knocked out) from the cell and the gene is selected from the group consisting of p50 (NFKB1), p52 (NFKB2), p65 (RELA), c-Rel (REL), and RelB (RELB). In some examples, the gene modification is CRISPR-Cas9 based. In some examples, there is provided a CRISPR-Cas9 based genetically modified cell as described herein.

In some examples, there is provided a p50 knockout cell line. In some examples, there is provided a p52 knockout cell line. In some examples, there is provided a p65 knockout cell line. In some examples, there is provided a c-Rel knockout cell line. In some examples, there is provided a RelB knockout cell line.

In some examples, the cell line may be a mammalian cell line. In some examples, the cell line may be a human cell line. In some examples, there is provided a genetically modified monocytic cell lines. In some examples, the cell may be a U937 cell. In some examples, there is provided a p50 knockout U937 cell line. In some examples, there is provided a p52 knockout U937 cell line. In some examples, there is provided a p65 knockout U937 cell line. In some examples, there is provided a c-Rel knockout U937 cell line. In some examples, there is provided a RelB knockout U937 cell line.

In the present disclosure, the inventors have generated five knock-out (KO) cell lines in U937 background e.g., p50-KO U937, p52-KO U937, p65-KO U937, Rel-KO U937 and RelB-KO U937.

In another aspect, there is provided a method of identifying a therapeutic target for treating acute myeloid leukemia (AML), the method comprising using the cell as described herein. In some examples, the cell may be one or more genetically modified knock out cells: p50-KO, p52-KO, p65-KO, cRel-KO, and/or RelB-KO.

In some examples, the present disclosure provides for a method of finding new AML targets using one or more genetically modified knock out cells: p50-KO, p52-KO, p65-KO, cRel-KO, and/or RelB-KO.

In some examples, the present disclosure provides for a method of finding new AML targets using genetically modified knock out U937 cells: p50-KO U937, p52-KO U937, p65-KO U937, cRel-KO U937, RelB-KO U937.

In yet another aspect, there is provided a method of treating acute myeloid leukemia (AML) in a subject in need thereof, comprising modulating/activating/increasing/improving the activity or expression of an NF-kB pathway.

In various embodiments, there is provided use of an agent/composition that is capable of modulating/activating/increasing/improving the activity or expression of an NF-kB pathway in the manufacture of medicament in treating AML.

In various embodiments, there is provided a method wherein the modulating/activating/increasing/improving of an NF-kB pathway modulates/regulates multiple pathways comprising a metabolic pathway, an inflammatory pathway, a cancer associated pathway, and combinations thereof.

In various embodiments, there is provided a method wherein the NF-kB pathway gene is p65.

In various embodiments, there is provided a method wherein the metabolic pathway includes, but is not limited to, oxidative phosphorylation (OXPHOS), mitochondrial dysfunction, Sirtuin signaling, mTOR signaling, and the like.

In various embodiments, the modulation/regulation (such as inhibition/decrease/reduction and/or increase/amplify) of metabolic pathway is determined by methods known in the art.

In some examples, the modulation/regulation (such as inhibition/decrease/reduction and/or increase/amplify) of metabolic pathways may be determined by measuring bioenergetic profiles.

In some examples, measuring of bioenergetic profiles may include but is not limited to measuring oxidative phosphorylation (OXPHOS), measuring extracellular acidification rate (ECAR) for lactate production, measuring glycolytic rates, measuring mitochondrial metabolic rates, measuring ATP production rates, and the like.

For example, oxidative phosphorylation is observed/analyzed/measured/determined by performing assays that determines oxygen consumption rate (OCR). In some examples, OCR may be measured using assays that determine/measure such as but is not limited to, cellular respiration rate, mitochondrial function, and the like. In some examples, OCR may be measured using assays such as, but is not limited to, mito stress assays, Seahorse analyzer assay, Oxygen Consumption Rate Assay Kits, and the like. In some examples, OCR is measured using mito-stress assays.

In some examples, glycolysis is observed/analyzed/measured/determined by performing assays that determines lactate production (which can be analyzed/measured/determined through extracellular acidification rate (ECAR)). In some examples, ECAR may be measured using assays such as, but is not limited to glycolysis-stress assays, Seahorse XF assays, Time-Resolved Fluorescence assay, and the like. In some examples, ECAR is measured using a glycolysis-stress assay.

In various embodiments, there is provided a method wherein the cancer associated pathway includes, but is not limited to pathways related to acute myeloid leukemia (AML), small lung cancer, pancreatic adenocarcinoma, and the like, optionally, the cancer associated pathway includes, but is not limited to, AML and AML-associated signaling pathways such as IL-6, IL-7, JAK, CXCR4, JAK-STAT, GM-CSF, and the like.

In some examples, AML associated pathogenesis may include markers such as but is not limited to CD244, CD123, CD117, CD45RA, SLCO2B1, SLC17A9, TCTN3, ULBP2, SLC43A1, CSF3R, CLEC7A, ABCA7, CD48, ATP13A2, SMAD3, SLC1A4, IL6R, HIST3H2A, PFDN2, SLC44A1, NACA, CPNE9, HECTD3, SLC30A5, RALGDS, CYCS, FHL2, PPIL3, CDKL5, GJB2, RPL6, RAB13, ADAMTS7, NDUFS4, HERPUD2, GLG1, MTHFR, PSMA2, TNNT1, TMEM14A, MCTS1, PDCL3, RPSA, ARL3, INPP5B, RPLPO, IL19, ZER1, RPL10, RPS2, VCL, SKA2, GGCT, RPS4X, VPS25, MGST1, VPS29, BTF3, HMOX1, RPL5, TMEM60, EXOC3, IER3, AVL9, RIC8A, YWHAB, GTSF1, RPL8, SLC25A26, GPT2, TCIRG1, TIMM9, MSH5, ADA, SEC11C, TIMM8A and the like.

In some examples, the methods as described herein causes the inhibition of one or more AML associated pathogenesis marker selected from the group consisting of CD244, CD123, CD117, CD45RA, SLCO2B1, SLC17A9, TCTN3, ULBP2, SLC43A1, CSF3R, CLEC7A, ABCA7, CD48, ATP13A2, SMAD3, SLC1A4, IL6R, HIST3H2A, PFDN2, SLC44A1, NACA, CPNE9, HECTD3, SLC30A5, RALGDS, CYCS, FHL2, PPIL3, CDKL5, GJB2, RPL6, RAB13, ADAMTS7, NDUFS4, HERPUD2, GLG1, MTHFR, PSMA2, TNNT1, TMEM14A, MCTS1, PDCL3, RPSA, ARL3, INPP5B, RPLPO, IL19, ZER1, RPL10, RPS2, VCL, SKA2, GGCT, RPS4X, VPS25, MGST1, VPS29, BTF3, HMOX1, RPL5, TMEM60, EXOC3, IER3, AVL9, RIC8A, YWHAB, GTSF1, RPL8, SLC25A26, GPT2, TCIRG1, TIMM9, MSH5, ADA, SEC11C, and TIMM8A.

In some examples, the method comprises detecting and/or determining the expression of the markers is one or more selected from the group consisting of ATP13A2, SMAD3, SLC1A4, IL6R, HIST3H2A, PFDN2, SLC44A1, NACA, CPNE9, HECTD3, SLC30A5, RALGDS, CYCS, FHL2, PPIL3, CDKL5, GJB2, RPL6, RAB13, ADAMTS7, NDUFS4, HERPUD2, GLG1, MTHFR, PSMA2, TNNT1, TMEM14A, and MCTS1.

In various embodiments, there is provided a method wherein the inflammatory pathway comprises TLR, TNFR2, CCL22, IL-10, CD40, IL-17A, PEDF, and/or IL-6 signaling.

In some examples, the method may include inflammatory pathways such as but is not limited to, TLR, TNFR2, IL-6, CCL22, IL-10, IL-17A, and the like.

In various embodiments, there is provided a method wherein the method regulates/modulates (such as inhibition/decrease/reduction) gene/protein expression/levels of a lysosomal associated protein.

In some examples, the method that regulates/modulates gene or protein activity/expression/levels (such as inhibition/decrease/reduction) of a lysosomal associated protein may include such as but is not limited to, knockdown using shRNA, siRNA, CRISPR-Cas9 shRNA, transcription activator-like effector nucleases (TALENs), zinc-finger nucleases (ZFNs), use of inhibitors, drugs, and the like. In some examples, the gRNAs that are used in CRISPR-Cas9 mediated knockdown are described in Table 1.

In some examples, the lysosomal-associated protein may comprise such as but is not limited to, lysosomal associated membrane protein (such as LAMP1, LAMP2, LAMP3), ATP13A2 (Park9), lysosome integral membrane protein 2 (LIMP2), and the like.

In some examples, the method may include downstream targets of lysosomal-associated proteins (such as ATP13A2/Park9) such as but is not limited to TFEB, phosphorylated TFEB (such as at ser-211), SYT11, α-synuclein and the like. In some examples, the downstream target of ATP13A2 (or Park9) is TFEB.

In various embodiments, there is provided a method wherein the method regulates/modulates (such as inhibition/decrease/reduction) the function of a lysosomal-associated protein.

In some examples, the method that regulates/modulates (such as inhibition/decrease/reduction) the function of a lysosomal-associated protein may comprise, but is not limited to, drugs, inhibitors, use of antibodies and/or aptamers, chemical genetics, analog-sensitive enzyme alleles, chromophore-assisted laser inactivation, and the like.

In some examples, lysosomal function is measured by methods known in the art such as but is not limited to analyzing lysosomal mass by determining/measuring/observing/analyzing the lysosomal-associated protein and/or downstream targets, analysing lysosomal acidification, and the like.

In some examples, the lysosomal-associated protein and/or downstream targets of lysosomal-associated protein may be measured/observed/analyzed by flow cytometric analysis, western blot, quantitative analysis of microscopy (such as confocal microscopy) images, histology staining, gene expression and the like.

In some examples, lysosomal acidification may be analysed by methods such as but is not limited to staining with lysotracker, use of ratiometric probe to measure lysosomal pH, and the like.

In various embodiments, there is provided a method wherein the lysosomal-associated protein is ATP13A2 (also known as PARK9).

In various embodiments, there is provided a method wherein the method comprises administering an agent that modulates/inhibits/reduces/decreases the activity or expression of ATP13A2 gene/mRNA/protein.

In some examples, reduction in AML pathology may include but is not limited to, reduction/elimination/loss of expansion/accumulation and/or proliferation of leukemia-stem cells (LSCs), reduction in colony forming ability, reduction/elimination/decreased induction of inflammatory proteins, reduction/elimination/decreased pathological changes, increased lysosomal mass and/or increased lysosomal acidification, reversal on impact on normal hematopoiesis, reduction of pro-leukemic cytokine signatures, reversal in dysfunctional lymphoid immune compartment/lymphoid depletion in organ (such as spleen), reduced weight loss, increased survival rate and the like.

In some examples, reduction/elimination/loss of LSC proliferation/expansion may be measured by methods known in the art, such as but is not limited to, xenograft expansion assay, cell proliferation assay, and the like.

In some examples, reduction/elimination/loss of LSC formation may be measured by methods known in the art, such as but is not limited to, colony-forming unit assay, methylcellulose colony-forming unit (CFU) assay (which determines the functional capability of a cell to form LSC colonies)), and the like.

In some examples, decreased induction of inflammatory proteins may include inflammatory proteins such as but is not limited to, TNFα, IL-6, IL-1β, IL-18, IL-17a, and the like.

In some examples, pathological changes may include but is not limited to, necrosis, cellularity in organs (such as bone marrow, spleen), apoptosis, and the like. In some examples, necrosis may include but is not limited to deposition of amorphous granular eosinophilic material, hemorrhages, and the like.

In some examples, cytokines and chemokines that are modulated in AML may include but are not limited to, CCL22 (MDC), CCL3, CCL4, IL-10, IL-5, IL-8, IL-1B, PDGF-AA, CXCL1, CXCL2, CXCL3, CXCL9, CXCL10, CXCL12, and the like. As shown in the experimental data of the present invention, levels of tumor promoting cytokine such as CCL22 (MDC) is increased, whereas the levels of anti-tumor cytokine IL-10 is decreased.

Without wishing to be bound by theories, it is believed that the inhibition of the ATP13A2 pathway advantageously rectifies energetic state of tumor cells to balance.

In various embodiments, there is provided a method wherein treatment reduces one or more indications such as but is not limited to, reduction/inhibition/decrease of oxygen consumption rate (OCR), reduction/inhibition/decrease/reversal in maximum/increased respiration, reduction/inhibition/decrease in spare respiratory capacity, reduction/inhibition/decrease of non-mitochondrial respiration, and the like.

In various embodiments, there is provided a method wherein treatment reduces one or more indications such as but is not limited to reduction/inhibition/decrease in glycolysis, reduction/inhibition/decrease in glycolytic capacity, reduction/inhibition/decrease/reversal in maximum/increased in mitochondrial respiration, and the like.

In various embodiments, there is provided a method wherein treatment reverses/improves dysfunctional mitochondrial function.

In various embodiments, there is provided a method wherein treatment reduces one or more indicators such as but is not limited to decreased/elimination/reduced of the ability to proliferate and/or form colonies, and the like.

In various embodiments, there is provided a method wherein the subject is further treated with inhibiting agents such as but is not limited to, a chemotherapeutic agent, an immune therapy agent, a cellular therapy agent, an oligonucleotide, an antigen binding molecule, a small molecule inhibitor, and/or combinations thereof, and the like.

In some examples, the immune therapy agent may include such as but is not limited to, small molecules, monoclonal antibodies, checkpoint inhibitors, cytokines, vaccines, chimeric antigen receptor (CAR) T cell therapy, and the like.

In some examples, the cellular therapy agent may include such as but is not limited to stem cell therapy, CAR T cell therapy, and the like. In some examples, stem cell therapy may include but is not limited to the use of embryonic stem cells, tissue-specific stem cells, mesenchymal stem cells, induced pluripotent stem cells, and the like.

In some examples, the oligonucleotide may include such as but is not limited to small single stranded nucleic acid, antisense oligonucleotide, RNAi, therapeutic oligonucleotide, and the like.

In some examples, the antigen binding molecule may include but is not limited to an antibody, T-cell receptors (TCR), major histocompatibility complex (MHC) class I and class II antigens (MHC class I and MHC Class II). In some examples, the antibody may include such as but is not limited to an intracellular antibody, an extracellular antibody, and the like.

In some examples, the small molecule inhibitor may include such as but is not limited to, tyrosine & serine/threonine kinases inhibitors, proteosomes inhibitors, specific inhibitor of ATP13A2, and the like.

In some examples, the inhibiting agent may reduce AML pathogenesis via direct interaction with a target (such as ATP13A2, TFEB) and/or indirect effects through deregulation of downstream components of a pathway (such as deregulated immune regulatory cytokines/chemokines). In some examples, deregulated cytokines may include but is not limited to MDC (CCL22), IL-10, IL1RA, PDGF-AA, FLT3-G, VEGF, and the like.

In various embodiment, the method comprises further treating the subject with a chemotherapeutic agent.

In various embodiments, the chemotherapeutic agent may include, but is not limited to Giltertinib, Cytarabine, Anthracycline, Venetoclax, Glasdegib, Ivosidenib, Enasidenib, and the like.

In some aspect, there is provided an ATP13A2 inhibiting agent for use in therapy or medicine.

In one aspect, there is provided an ATP13A2 inhibiting agent for use in treating AML.

In one aspect, there is provided use of an ATP13A2 inhibiting agent in the manufacture of a medicament for treating a disease (such as AML).

In various embodiments, the method may comprise testing/assessing the therapeutic potential of shRNA, siRNA, antisense oligonucleotides (ASOs) in inhibiting ATP13A2 in AML. In some examples, antisense oligonucleotides may include but is not limited to small single-stranded nucleic acids of multiple chemistries with clinical utility for multiple indications, and the like.

In various embodiments, the method may comprise designing ATP13A2 specific shRNA/ASOs in gapmer configuration and/or testing the inhibitory efficacy of leukemic stem cell (LSC) functions and metabolic adaptation of RelA deficient AML cells.

In various embodiments, the method may comprise testing the most effective shRNA/ASOs for off-target toxicity and immune response. In various embodiments, the present disclosure provides for ASOs for off-target toxicity and immune response.

In various embodiments, the present disclosure provides for ASOs for in vivo application to improve one or more of the following: hematopoiesis, induced pathological changes, and survival through inhibition of xenograft expansion in a humanized mice model.

In various embodiments, the method may comprise analyzing and validating in a humanized PDX model the ASOs showing the best therapeutic benefits in the CDX model.

DNA Constructs and Paired Guide RNAs (pgRNAs) Cloning in Lentiviral Vector

In some examples, there is provided a method of knocking out a gene from a cell line. In some examples, the method comprises introducing a paired guide RNA (gRNA) cloning vector and an expression vector into a cell line. In some examples, the expression vector may be a lentiviral expression vector. In some examples, the pair of gRNA may comprise a set of reverse complementary forward and reverse oligomers for targeting the gene of interest (e.g. targeting NF-kB family of transcription factors). In some examples, the method of knocking out a gene from a cell line utilises CRISPR-Cas9 technique.

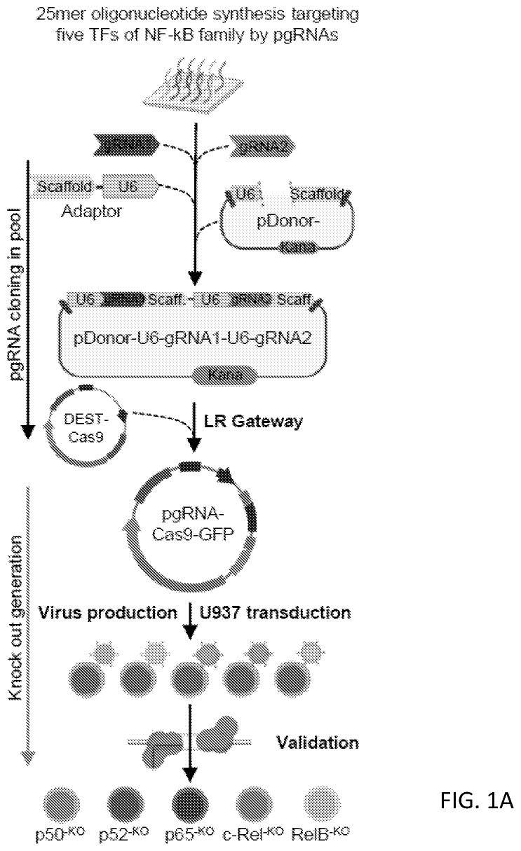

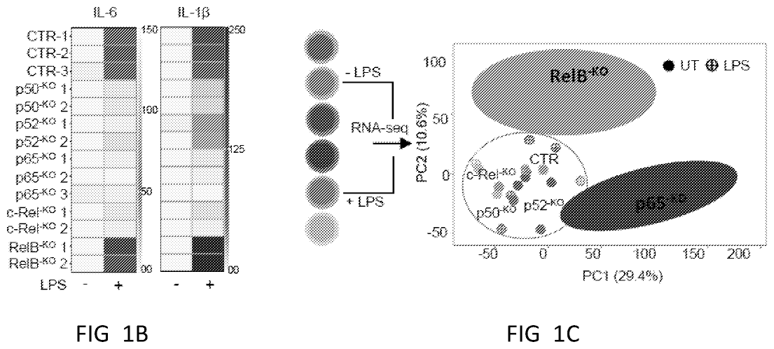

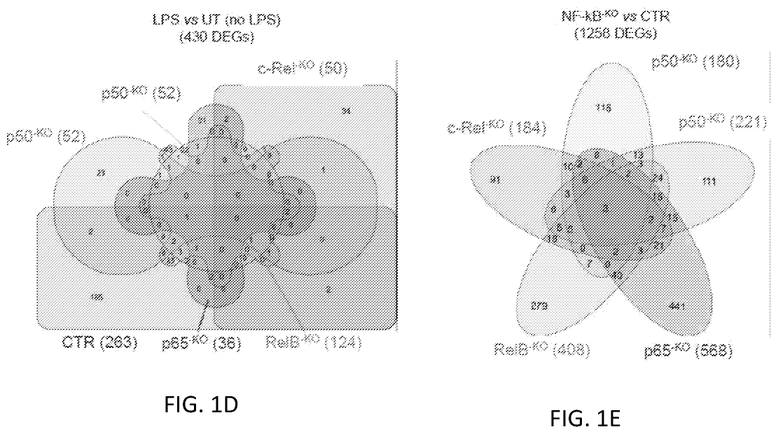

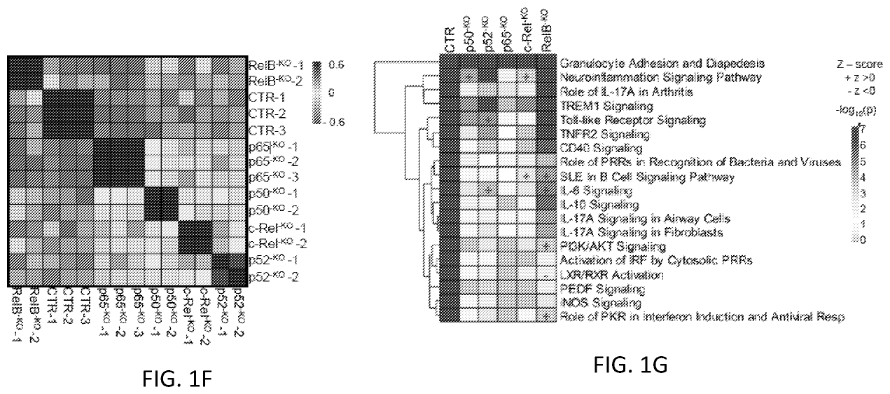

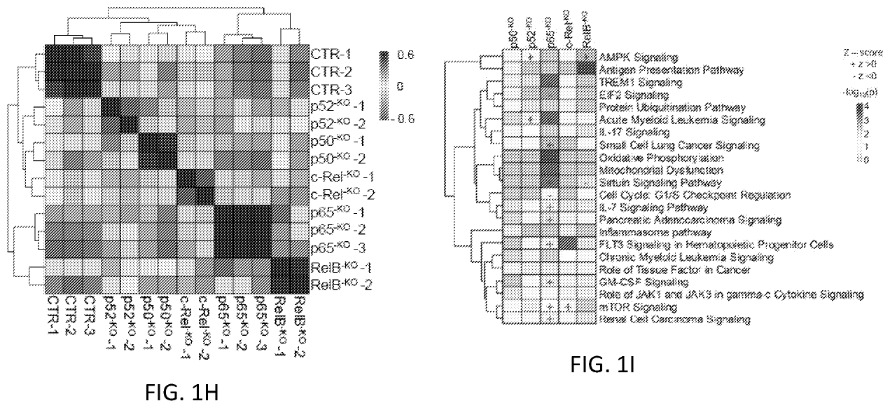

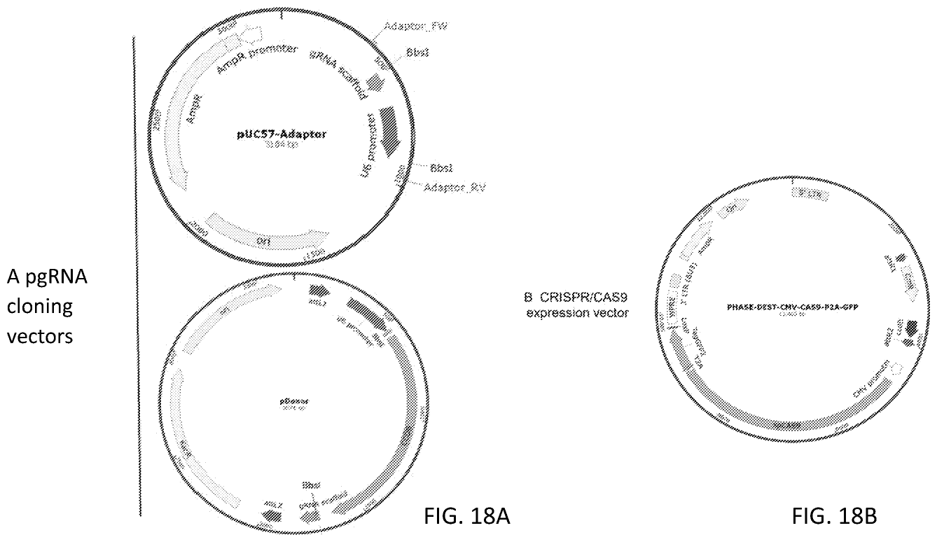

In some examples, there is provided a method of knocking out a gene from a cell line, wherein the method is as described in FIG. 1A. In some examples, the present disclosure comprises an oligomer based CRISPR-Cas9 approach to generate KOs-based on two sets of plasmids: paired gRNAs cloning vectors (for example pAdaptor and pDonor) and an expression vector (for example a lentiviral expression vector such as, but not limited to, PHASE-DEST-CAS9-T2A-GFP) (examples as shown in FIG. 1A, FIG. 18A and FIG. 18B)

In some examples, for the cloning of paired gRNAs into pDonor vector, the method comprises providing a set of reverse complementary forward and reverse oligomers for each gRNA targeting NF-kB family of transcription factors (TFs) that were synthesized from IDT™ (Integrated DNA Technologies™). In some examples, the oligomer may be about 20 to 30 bases long. In some examples, the oligomer may be about 21, 22, 23, 24, 25, 26, 27, 28, 29, or 30 bases long. In some examples, the oligomer may be 25 bases long. In some examples, as shown in FIG. 1, the method comprises first cloning the pgRNAs into the adaptor vector in pool using gRNAs specific reverse complementary forward and reverse primers (such as primers provided in Table I), that involves equimolar mixing of forward and reverse primers, phosphorylation, followed by annealing and ligation to the bbsl digested adaptor (adaptor+pairs of guide RNAs) (FIG. 1 and FIG. 18A).

In some examples, the method comprises pooling and purifying the ligated pgRNAs to the adaptor (such as for example by AMPure beads at 1:1 ratio). In some examples, the method comprises further ligating the pgRNAs to an enzyme digested donor plasmid (e.g., Bbsl digested pDonor), followed by transformation in a host cell (such as in TOP10 cells) and plating onto selection plates (such as Kanamycin selection plates; as shown in FIG. 1A). In some examples, the method comprises selecting at least twice more or three times more, or four times more, or five times (or more) more bacterial clones than paired guide RNAs to confirm the representation of paired guide RNAs in pool cloning by sequencing (such as Sanger sequencing). In some examples, the method comprises identifying target specific paired guide RNAs based on sequencing (such as Sanger sequencing). In some examples, the method comprises further shuttling guide RNAs efficiently to the DEST containing lentiviral CAS9 expression plasmid using LR-gateway reaction according to the experimental need (as shown in FIG. 1A, FIG. 18B).

### Lentivirus Production, Infection and Knockouts

In some examples, the method comprises producing viral particles by co-transfection of plasmids (such as lentiviral expression plasmids containing paired gRNAs and wild type CAS9, dCAS9 derivatives) together with other plasmids (such as packaging plasmids) in a cell line (such as Lenti-X cell line) using transfection reagent (such as Xfect polymer) in cell culture plates (such as 6 well plates). In some examples, the method comprises collecting viral supernatants post transfection (such as 72 h post transfection) and filtered using a filter (such as 0.45 μm filter) followed by concentration using a concentrator (such as LentiX concentrator). In some examples, the method comprises transducing the cells with a concentration of viral particles (such multiplicity of infection (MOI) of 10) in the presence of a cationic polymer (such as polybrene, 2200g for 1 h at 22° C.). In some examples, the method comprises cloning (such as single cell cloning) to identify clones (such as productive knock out clones) post infection (such as 5 days post infection), where cells were directly sorted into cell culture plates (such as round bottom 96 well plates) using fluorescent protein (such as constitutively expressed GFP). In some examples, the method comprises expanding clones (such as single cell clones) for two weeks, with cells (such as 90% expanded cells) from the cell culture plate (such as 96 well plate) transferred into a new cell culture plate (such as V shaped 96 well plate) and pelleted by centrifugation followed by lysis of cell pellet with buffer (such as 50 mM NaOH, 96° C., for 30 min) followed by buffer (such as NaOH) neutralization by addition of a buffer (such as 10% 1M Tris HCl pH 8.0). In some examples, the method comprises centrifuging (such as 4.5 k for 5 min) the cell culture plate and collecting the supernatant containing nucleic acid (such as genomic DNA) to be used for the validation of genetic editing (such as by PCR amplification). In some examples, the method comprises validating genetic editing by amplifying and sequencing targeted regions using specific sets of primers. In some examples, the method comprises based on sequencing (such as Sanger sequencing), selecting clones (such as a minimum of two clones) with editing for each target gene to validate for the loss at protein level (such as using western blotting).

### TLR Stimulation and Cytokine Detection

In some examples, the method comprises culturing the cells in medium (such as RPMI) supplemented with serum (such as 10% FCS), amino acid (such as glutamine), antibiotics (such as penicillin, streptomycin) and salt (such as Na-Pyruvate). In some examples, the method comprises seeding cells in cell culture plates (such as 12 well plates in two sets) on the day of protein stimulation (such as TLR stimulation) and allowed to rest (such as for 2 h). In some examples, the method comprises stimulating the cells with bacterial components (such as LPS, after 2 h). In some examples, the method comprises lysing the cells in the first set post protein stimulation (such as 6 h post LPS stimulation) in chemical solution (such as trizol) for nucleic acid (such as RNA) isolation, and second set of stimulation (such as for 24 h), with supernatants collected and protein induced cytokines (such as LPS induced cytokines: IL-6, IL1-β) analysed using a kit (such as Ready-SET-Go Elisa Kit).

### RNA Sequencing Data Analysis

In some examples, the method comprises assessing the reads obtained from sequencing (such as Illumina sequencing) for quality using a software (such as FASTQC version 0.11.7). In some examples, the method comprises using a software (such as Salmon version 0.11.3) for quantification (such as quasi-mapping-based quantification) of transcript abundances from the paired end reads where the reference set of human transcripts was obtained from a software (such as Gencode version 29). In some examples, the method comprises summarizing transcript level counts generated by the software (such as Salmon) to gene-level counts using a software (such as tximport R/Bioconductor package version 1.2.3). In some examples, the method comprises importing the gene counts into a software (such as DESeq2) for the analysis of differentially expressed genes (DEGs). In some examples, the method comprises fitting a model (such as negative binomial generalized linear model (GLM)) to the counts data which included coefficients to model the effect of tissue type, treatment and sample batch. In some examples, the method comprises estimating parameters (such as size factors, dispersion parameters) in the model (such as GLM) from counts data. In some examples, the method comprises adjusting the counts by a method (such as median ratio method) to normalize for library sizes. In some examples, the method comprises performing a test (such as a Wald test) on model coefficients to identify DEGs. In some examples, the method comprises selecting the DEGs that fulfil criteria of nominal p-value <0.1 (such as nominal p-value <0.005), at least 1.5-fold change, and a baseMean of more than 10). In some examples, the method comprises analyzing the biological pathways and functions enriched in the DEGs using a software (such as Ingenuity Pathway Analysis (IPA)). In some examples, the method comprises writing custom scripts in a software (such as R) to perform analysis (such as principal component analysis (PCA)) and to analyze correlations between samples. In some examples, the method comprises expressing the normalized gene abundances as reads (such as log transformed transcripts per million mapped reads, log 2 (TPM+1.0)), where a pseudocount value (such as 1.0) was added to prevent negative values, and only genes with baseMean above a value (such as 10) and having a gene ID (such as Entrez gene ID) and symbol were used. In some examples, the method comprises using a software (such as R package FactoMineR) for analysis (such as PCA) and another software (such as pheatmap) for drawing heatmaps. In some examples, the method comprises evaluating the sample correlations with a statistical method (such as Spearman's correlation) using a function (such as cor.test function) of a software (such as R base library).

Analysis of ChIP-ChIP Data from Studies Known in the Art

In some examples, the method comprises analysing the binding of transcription factors (such as NF-kB transcription factors) to the promoters of differentially expressed genes, by obtaining from a database (such as ArrayExpress) with accession ID (#E-WMIT-6) the data from a previous ChIP-ChIP experiment (such as NF-kB ChIP-ChIP) on U937 cells. In some examples, the method comprises processing the raw data (such as raw two-color microarray data) using a software (such as marray Bioconductor package in R) to perform background correction and normalization (such as within array normalization) by a method (such as loess method). In some examples, the method comprises normalizing (such as quantile normalizing) the resultant signal intensities across all arrays in the dataset. In some examples, the method comprises calculating the final signal intensities (such as final ChIP signal intensities) as average of a number of samples (such as triplicates). In some examples, the method comprises an assessment (such as metabolic assessment) through analysis (such as extracellular flux analysis): analysis of rates (such as oxygen consumption rate (OCR), and extracellular acidification rate (ECAR)). In some examples, the method comprises hydrating a plate (such as utility plate) with sterile water (such as overnight, 37° C., non-CO₂ incubator). In some examples, the method comprises incubating a tube (such as Falcon tube) with buffer (such as calibration buffer) (such as overnight, 37° C., non-CO₂ incubator). In some examples, the method comprises seeding cells (such as U937 controls and p65-KO cells) in cell culture plate (such as 6 well plate) the next day (such as for 6 h) and measuring following the incubation (such as after 6 h) the rates (such as OCR and ECAR) using an instrument (such as (XFp analyzer). In some examples, the method comprises seeding cells (such as U937 control and p65^{-KO} cells) on polymer coated (such as poly-L-lysine coated) cell culture plates (such as XFp Seahorse plates). In some examples, the method comprises replacing the cell culture medium with a medium (such as XF base medium) supplemented with amino acids (such as 2 mM glutamine (Gln), 11 mM glycolic acid (Glc)) and chemical compound (such as 2 mM pyruvate (Pyr)) with an adjusted pH (such as pH 7.4). In some examples, the method comprises incubating cells (such as 37° C. and 5% CO₂ for one hour). In some examples, the method comprises injecting compounds (such as oligomycin at 1.5 μM, ptrifluoremethoxyphenylhydrazone (FCCP, 0.5 μM), and a mixture of antimycin A (0.5 μM) and rotenone (0.5 μM)) from a kit (such as XFp cell mito stress test kit) during the assay. In some examples, the method comprises the reflection of the spare respiratory capacity of a cell or the maximum respiratory rate that can be reached by the increase of a rate (such as OCR) after application of a compound (such as FCCP). In some examples, the method comprises verifying a rate (such as ECAR rate) through application of a test kit (such as glycolysis stress test kit). In some examples, the method comprises supplementing the test kit assay medium (such as glycolysis stress test kit assay medium) with an amino acid (such as 2 mM Gln). In some examples, the method comprises adding a drug (such as 1 μM oligomycin) followed by another drug (such as 50 mM 2-deoxyglucose) after manual injection of a sugar (such as 10 mM glucose). In some examples, the method comprises the allowance for correlation of an increase in rate (such as ECAR) with a higher rate (such as glycolytic rate) through the injection of a sugar (such as glucose) to the medium (such as so far glucose-free medium). In some examples, the method comprises calculating a capacity (such as glycolytic capacity) of a cell, which is the maximum rate (such as maximum glycolytic rate) that can be achieved after application of a drug (such as oligomycin). In some examples, the method comprises using a software (such as Agilent Seahorse Software Wave 2.3) for data analysis. In some examples, the method comprises generating a test report (such as the seahorse XF cell energy phenotype test report) through a report generator (such as wave 2.3 report generator) using the assay result data from a test (such as seahorse XF cell mito stress test).

### Methylcellulose Human Colony-Forming Unit (CFU) Assay

In some examples, the method comprises transferring a medium (such as MethoCult optimum medium-without EPO) from freezer temperature (such as −20° C.) to fridge temperature (such as 2-8° C.) the day before plating cells for an assay (such as CFU assay). In some examples, the method comprises seeding cells (such as U937 controls and p65^{-/-} cells in complete medium) in each well of a cell culture plate (such as 12 well plate) (such as for 6 h). In some examples, the method comprises following incubation (such as 6 h incubation) either washing the cells with a wash medium (such as IMDM with 25 mM HEPES) and resuspended to make a more concentrated cell suspension (such as 10× cell suspension) for the untreated cells, or stimulated with cytokines (such as IL-6 for 12 h) followed by washing with a wash medium (such as IMDM with 25 mM HEPES) and resuspended to make a more concentrated cell suspension (such as 10× cell suspension). In some examples, the method comprises shaking a thawed medium (such as MethoCult medium) vigorously (such as for 1-2 minutes) and then let stand (such as for at least 5 minutes), until all bubbles rise to the top, before aliquoting. In some examples, the method comprises preparing medium aliquots (such as Methocult medium aliquots into 14 mL) using a needle (such as 16 gauge blunt-end needle). In some examples, the method comprises adding cell suspension (such as 100 μl (500 or 1000 cells) of cell suspension/plate) directly to pre-aliquoted tubes of complete medium (such as MethoCult medium) and mixed gently to make a homogeneous cell suspension. In some examples, the method comprises plating the cell suspension in medium (such as MethoCult) to a dish (such as 35 mm dish) in replicates (such as triplicate) for each clone (such as KO clone) and incubated (such as at 37° C., in 5% CO₂ with >95% humidity for 10 days). In some examples, the method comprises scanning the colonies using an instrument (such as EVOS M7000) and counted manually.

shRNA Knockdown

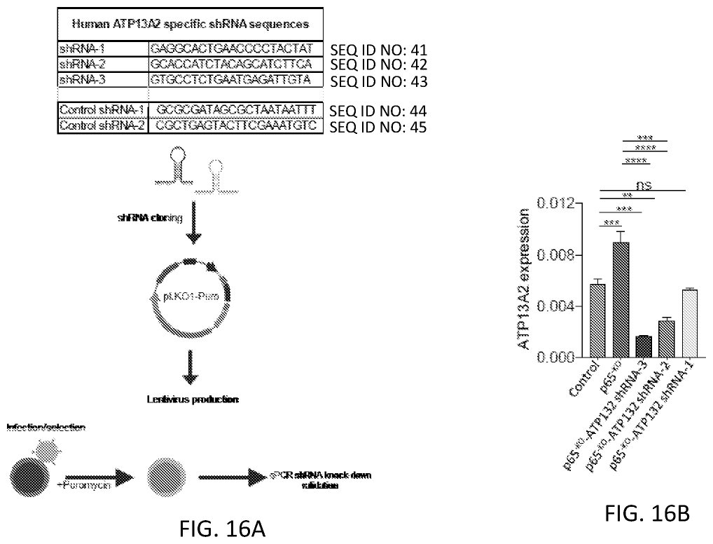

In some examples, the method comprises performing knockdown of a protein (such as ATP13A2) in cells (such as U937 and p65-KO cells) using a knockdown strategy (such as pLKO based shRNA strategy) transduced using a vector (such as lentiviral shRNA pLKO vector, as shown in FIG. 16A). In some examples, the method comprises selecting the transduced cells with antibiotics (such as 1 g/ml puromycin for 10 days). In some examples, the method comprises validating the knockdown (such as shRNA knockdown) efficiency using nucleic acid isolation (such as qPCR RNA isolation) and quantitative amplification reaction (such as qRT-PCR). In some examples, the method comprises isolating nucleic acid (such as total mRNA) from cells using a kit (such as RNeasy Mini Kit (Qiagen)) and reverse transcribed according to the instruction in the manual. In some examples, the method comprises performing an amplification reaction (such as realtime PCR) with nucleic acid (such as cDNA) and oligonucleotide primers in an instrument (such as AB Biosystem 7000). In some examples, the method comprises using conditions (2 min, 50° C., and 10 min, 95° C., followed by 40 cycles of 15 s, 95° C. and 1 min, 60° C. in 15 μl reactions) of an amplification reaction for an instrument (such as LightCycler).

### AML Xenograft Study

In some examples, the method comprises performing xenograft experiments (such as AML xenograft experiments). In some examples, the method comprises sub lethally irradiating (such as X-irradiated (1.2 Gy)) an animal (such as 4- to 6-week-old NOG mice) before transplantation (such as two days before). In some examples, the method comprises injecting cells (such as GFP+ CTR and p65^{-KO} U937 cells) into the animal (such as mice) via an organ (such as a tail vein) in a volume of buffer (such as PBS). In some examples, the method comprises generating humanized animal (such as humanized NOG mice) by sub lethally irradiating (such as X-irradiation (1.2 Gy) of an animal (such as 7 to 8 weeks old NOG mice) followed by injection of an organ (such as tail vein injection) of human blood (such as CD34+ human cord blood) derived from stem cells (such as hematopoietic stem cells (HSCs))/an animal (such as mouse). In some examples, the method comprises confirming successful engraftment by analysis (such as flow cytometric analysis) of cells (such as hCD45+ vs mCD45+) cells in the blood (such as peripheral blood) at different time points post human stem cells transplant (such as 4, 8, and 12 weeks post human HSC transplant). In some examples, the method comprises sub-lethally irradiating an animal (such as mice) post human stem cell transplant (such as 12 to 14 weeks post human HSC transplant) and after cells (such as U937 cells) were injected (such as after two days) as described for the xenograft (such as NOG xenograft). In some examples, the method comprises recording the weight of the animal (such as mice) daily. In some examples, the method comprises collecting blood (such as peripheral blood post cell injection (such as 5 to 7 and 11 to 16 days post AML− cells injection)) from an area of the animal (such as retro orbital sinus) using tubes (such as heparinized capillary tubes) for xenograft analysis. In some examples, the method comprises collecting a sample (such as bone marrow) at endpoint. In some examples, the method comprises performing the phenotyping of xenografts in the sample (such as in blood and bone marrow).

p65-p65 Dimer Regulated Gene Survival Analysis in AML Datasets

In some examples, the method comprises obtaining data (such as gene expression and survival data) from a database (such as cBioPortal) for datasets (such as AML datasets TARGET and TCGA). In some examples, the method comprises selecting only patients that went through standard chemotherapy (such as standard chemotherapy without BM transplantation), and additionally excluding samples (such as PML-RAR samples) from the analysis due to its interaction with a transcription factor (such as p65). In some examples, the method comprises performing an analysis (such as univariate Cox regression analysis) for the transcription factor regulated genes (such as p65 regulated genes) in each of the datasets (such as each of the two datasets). In some examples, the method comprises conducting analysis (such as random effects model meta-analysis) of the ratio (such as hazard ratio) to obtain a combined ratio (such as combined hazards ratio) depicted as plots (such as forest plots). In some examples, the method comprises of an analysis (such as meta-analysis) that reveals a list of genes (such as 29 genes) from which panels of all possible genes (such as 3, 4 genes) were constructed. In some examples, the method comprises using each of the panels in an analysis (such as multivariate Cox regression analysis) for each of the datasets (such as three dataset) to determine the predictive performance of the panels as indicated by its likelihood statistical value (such as ratio test P value). In some examples, the method comprises using a statistical approach (such as rank sum approach) to rank the panels using the results from the datasets (such as three datasets) and a combined statistical value (such as combined P value) computed using a statistical method (such as Fisher's method). In some examples, the method comprises performing analysis using a software (such as R version 3.6.2) using a package (such as survival and metafor packages) for statistical analysis (such as Cox regression analysis) and analysis of ratios (such as meta-analysis of hazard ratios) respectively.

### Results

Functional Delineation of NF-kB-TFs Identified p65 Mediated Suppression of Genes Associated with Metabolic Plasticity and Cancer Pathways

NF-kB family of transcription factors (TFs) exert their cellular and pathological functions through transcription regulation of a diverse set of target genes. Using U937 as an AML human cellular model, the inventors of the present disclosure systematically and comprehensively studied the cellular and pathophysiological functions of NFκB TFs in AML pathogenesis. All five NF-kB family of TFs genes; p50 (NFKB1), p52 (NFKB2), p65 (RELA), c-Rel (REL) and RelB (RELB) were knocked out from AML cell line U937 using an established CRISPR-Cas9 approach (FIG. 1A) and specific gRNAs (Table I).

TABLE 1
gRNAs targeting different genes

| S. | | | Targeted | | |
|---|---|---|---|---|---|
| Nr. | Gene | Species | Exon | gRNA-A | gRNA-B |
| 1 | NFKB1 | Human | Exon | TGTTCAGGCCTTCC | TATCACCATGGAATT |
| | | | 1 | CAAATA (SEQ ID NO: | CATGC (SEQ ID NO: |
| | | | | 1) | 2) |
| 2 | NFKB1 | Human | Exon | CAGGTAGTCCACCA | GAACAAGAAGTCTT |
| | | | 4 | TGGGAT (SEQ ID NO: | ACCCTC (SEQ ID NO: |
| | | | | 3) | 4) |
| 3 | NFKB2 | Human | Exon | GTAGCAACTCTCCA | GGGCAGGCGGCAT |
| | | | 1 | TGTCTC (SEQ ID NO: | GACTCAC (SEQ ID |
| | | | | 5) | NO: 6) |
| 4 | NFKB2 | Human | Exon | CAATAATACCATCCA | TGAACTTGCTGACC |
| | | | 2 | GACCC (SEQ ID NO: | TGTTTC (SEQ ID NO: |
| | | | | 7) | 8) |
| 5 | RELA- | Human | Exon | GACCCCGGCCATGG | CGCGGGCCCAGCT |
| | 1 | | 1 | ACGGTG (SEQ ID NO: | GCGACCC (SEQ ID |
| | | | | 9) | NO: 10) |
| 6 | RELA- | Human | Exon | TTCCCCCTCATCTTC | TCCTTGTGAGAATG |
| | 2 | | 2 | CCGGC (SEQ ID NO: | GGCATT (SEQ ID NO: |
| | | | | 11) | 12) |
| 7 | RELB- | Human | Exon | CCCGCGTGCATGCT | TTACCTAAGGCCCC |
| | 1 | | 1 | TCGGTC (SEQ ID NO: | CAGCTC (SEQ ID NO: |
| | | | | 13) | 14) |
| 8 | RELB- | Human | Exon | GTACTCGTCGATGA | GCCACGCCTGGTGT |
| | 2 | | 4 | TCTCTG (SEQ ID NO: | CTCGCG (SEQ ID NO: |
| | | | | 15) | 16) |
| 9 | REL- | Human | Exon | TGAACACTCACCGG | CGCGCCCCATGAAC |
| | 1 | | 1 | AGGCCA (SEQ ID NO: | ACTCAC (SEQ ID NO: |
| | | | | 17) | 18) |
| 10 | REL- | Human | Exon | ATCAGCAGGCAGCA | GGTCTATTACCTGG |
| | 2 | | 2 | TTCCAG (SEQ ID NO: | ATAGAA (SEQ ID NO: |
| | | | | 19) | 20) |

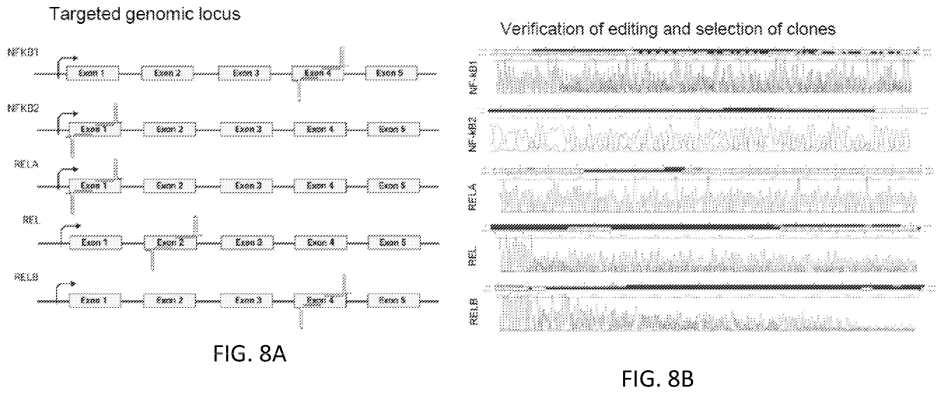

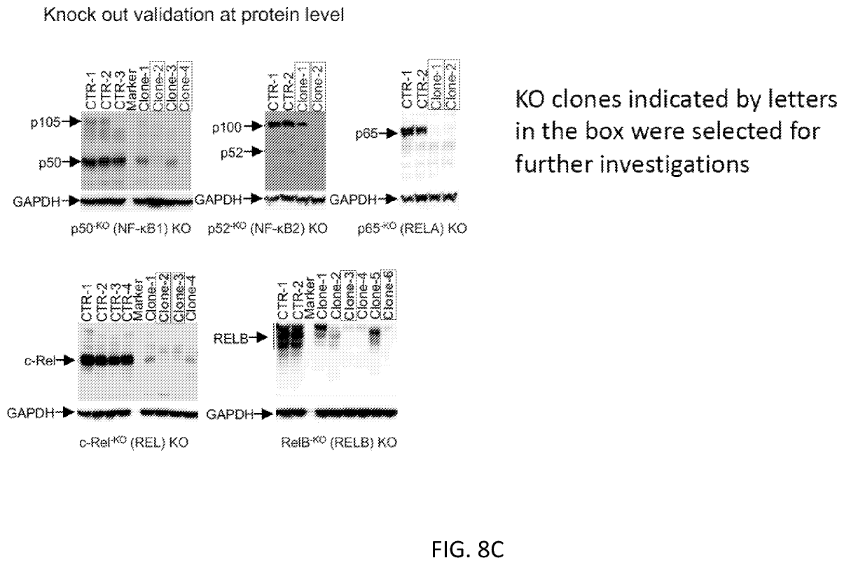

FIG. 8A to 8C show the validation of CRISPR/Cas9 KOs of NF-kB TFs. A total of 11 independent and validated KO clones representing each of five NF-kB TFs (i.e., p50, p52, p65, c-Rel and RelB) (FIG. 8A to 8C) were subsequently evaluated for potential loss of biological function through analysis of LPS mediated induction of inflammatory proteins. Upon LPS stimulation, the inventors of the present disclosure found impairment in the LPS-induced expression of secreted IL-6 and IL-13 proteins in all KO clones of p50, p52, p65 and c-Rel, compared to unedited cells (CRISPR-Cas9 targeted without any editing, Controls, CTR) (FIG. 1B). As expected, no effects were observed in RelB-KO clones (FIG. 1B). Overall, this shows that the NF-kB TF KO clones lost the targeted TF protein (FIG. 8C), but also the associated biological functions.

To gain mechanistic insights into the factor-specific functions of individual NF-kB TFs in AML pathologies, RNA-seq was performed on a total of 28 samples representing untreated and LPS-treated CTR (n=3 clones) and KO cells, p50^{-KO} (n=2 clones), p52^{-KO} (n=2 clones), p65^{-KO} (n=3 clones), c-Rel^{-KO} (n=2 clones) and RelB^{-KO} (n=2 clones) (FIG. 1C, left panel). Altogether, 548.4 million paired end 2×150 bp reads were obtained by Illumina sequencing of mRNAs extracted from these samples with a median of 19.0 million reads per sample. Of all sequenced reads, 94.8% (519.8 million) were ≥q30 on the Phread scale and 87.3% (478.9 million) mapped to known human transcripts. After obtaining read counts over genes, we selected 12,845 genes that were annotated with a unique Entrez gene id and symbol and were expressed well within detection limits with at least 10 read counts per sample on average after median ratio normalization of library sizes. The inventors of the present disclosure performed a principal component analysis (PCA) of the expression profiles of all 28 samples represented by these 12,845 genes (FIG. 1C, right panel). Samples plotted on PC1 and PC2 axes showed distinct clusters of p65^{-KO} and RelB^{-KO} clones, indicating their unique characteristics in comparison with the other NF-kB TF KOs which remained prominent in the context of pro-inflammatory LPS treatment (FIG. 1C, right panel).

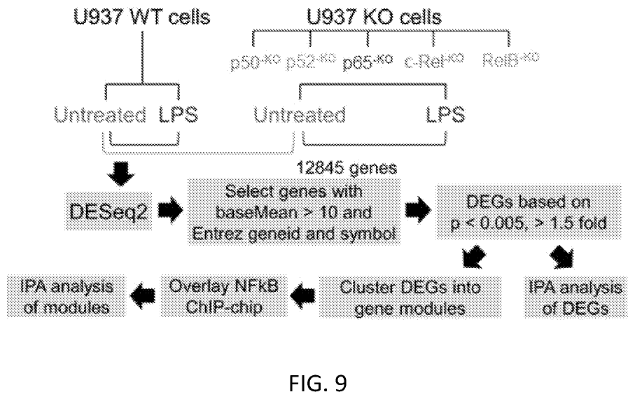

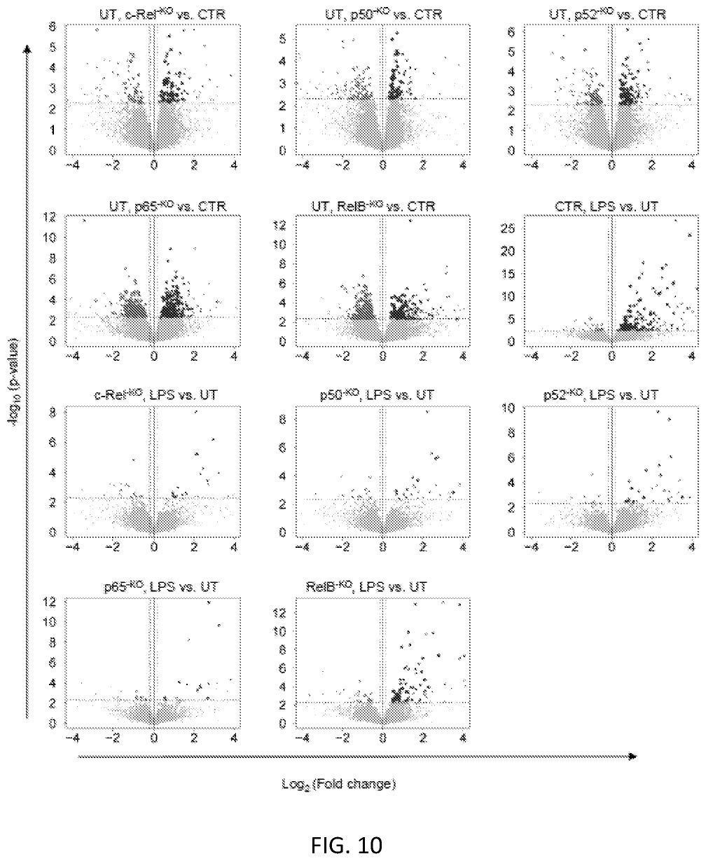

To better understand potential factor specific uniqueness, the inventors of the present disclosure performed differential gene expression (DEG) analysis using DESeq2 contrasting NF-kB KO vs. CTR cells and LPS treated vs. untreated cells (FIG. 9). In total 430 genes were found to be modulated by LPS treatment in CTR or NF-kB KO subclones based on p-value <0.005 and more than 1.5-fold changes in expression (FIG. 1D, FIG. 10). More genes were modulated by LPS in CTR cells (263 DEGs) than was seen in the individual NF-KB KOs (c-Rel^{-KO}, 50 genes; p50^{-KO}, 52 genes; p52^{-KO}, 50 genes; p65^{-KO}, 36 genes; RelB^{-KO}, 124 genes), suggesting an impaired inflammatory response in the NF-kB KOs. A Spearman's correlation analysis among 14 LPS treated samples, based on these 430 DEGs, showed that independent clones of the same NF-kB TF KO were clustered together and thus shared the highest correlation (FIG. 1F). Moreover, there were two major clusters, one comprising of p65^{-KO}, p50^{-KO}, c-Rel^{-KO} and p52^{-KO}, KO clones, and the second was comprised of the RelB^{-KO} and CTR cells, which respectively represent the canonical and non-canonical aspect of NF-kB pathways. The close correlation of RelB^{-KO} with CTR cells suggests a minor role of RelB in LPS induced inflammation that is consistent with earlier reports. Ingenuity pathway analysis (IPA) of the 430 DEGs among WT and—F-KB KO cells showed a severe loss in the ability of p50^{-KO}, p52^{-KO}, p65^{-KO} and c-Rel^{-KO} cells to induce activation of inflammatory pathways including TLR, TNFR2, and IL-6 signalling, in response to LPS (FIG. 1G). In contrast, in RelB^{-KO} cells the inflammatory pathways were only moderately impacted and responded almost in a similar manner to that seen in the CTR cells.

Since the major aim of the invention of the present disclosure was to investigate the pathophysiological functions of NF-kB TFs in AML, the inventors of the present disclosure identified p50, p52, p65, c-Rel and RelB TFs specific gene signatures through comparing the gene expression profiles of NF-kB KO cells with CTR cells in the steady unstimulated state (FIG. 1E). A total of 1258 DEGs were obtained based on p-value <0.005 and a more than 1.5-fold change in expression. An analysis of Spearman's correlation between the 14 untreated samples based on the 1258 DEGs showed independent clones of the same knockout clustered together with high correlation (FIG. 1H). Two broad clusters were observed, one included CTR, p50^{-KO}, p52^{-KO} and c-Rel^{-KO} cells, while the second comprised p65-KO and RelB-KO cells (FIG. 1H). The clustering of p65^{-KO} with RelB^{-KO} in non-stimulated cells could be due to a previously described functional interaction between p65 and RelB. IPA analysis of the DEGs in different NF-kB KO cells showed an enrichment of two main categories of pathways—metabolic (including Oxidative phosphorylation, mitochondrial dysfunction, Sirtuin and mTOR signalling) and cancer (including Acute myeloid Leukaemia, small lung cancer and pancreatic adenocarcinoma signalling) associated pathways (FIG. 1I). These pathways were overrepresented by positive z-score values in the p65^{-KO} cells (FIG. 1I). Taken together global gene expression analysis of NF-kB KO human AML cell line U937 suggests the state-specific homo- and heterogeneity among transcriptional regulation by NF-kB family of TFs.

### Deregulated p65 Specific Regulatory Network Leads to Metabolic Plasticity

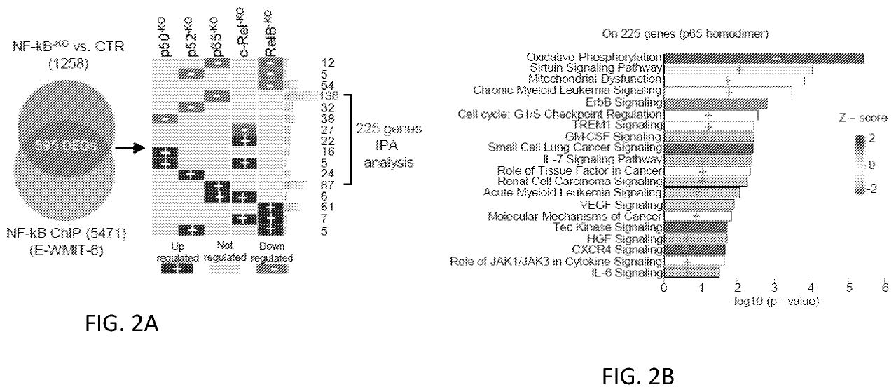

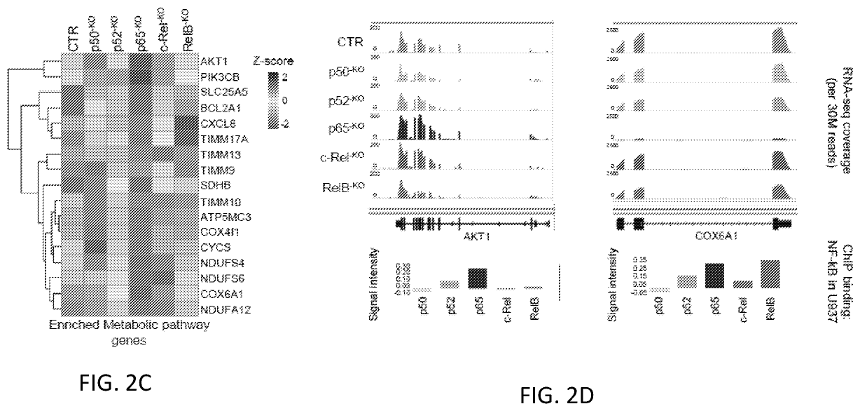

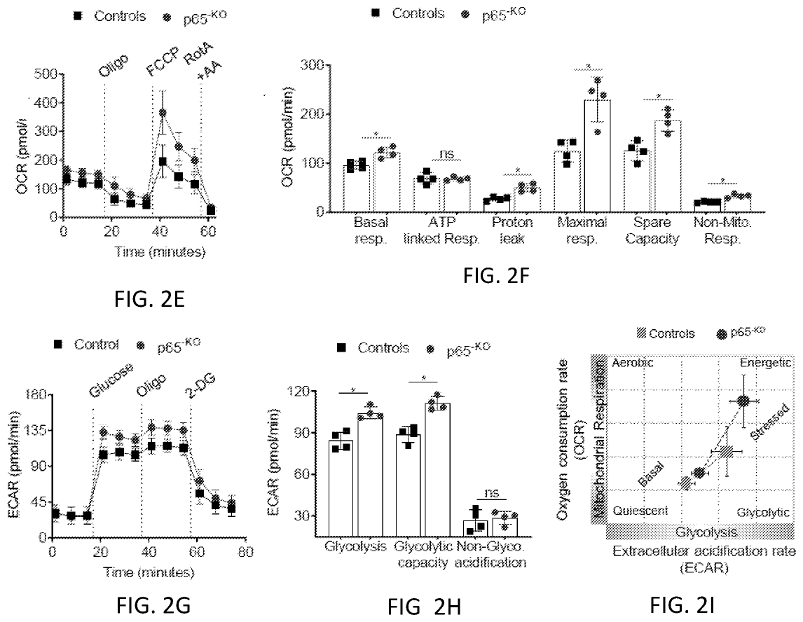

Regulation of gene expression by NF-kB TFs could occur either by direct binding to target DNA regions or through in an indirect manner. FIG. 2A to 2I shows that NFkB-p65 deficiency promotes metabolic adaptation in AML cells. In order to identify direct targets of NF-kB in the data set of 1258 DEGs (in steady state, FIG. 1E) from the present disclosure, the inventors of the present disclosure intersected this list with a known in the art ChIP-ChIP NF-kB binding profile described in U937 cells (FIG. 2A, left panel). This identified 595 genes that are direct targets of NF-kB TFs. These 595 genes were clustered in a total of 22 gene modules suggested to be directly regulated by NF-kB homodimers or heterodimers (≥5 genes/cluster, FIG. 2A, right panel). Among these modules, the p65/p65 homodimer specific two modules (with 138 downregulated and 87 upregulated genes, respectively; total 225 genes) appeared to be the most prominent (FIG. 2A, right panel). These 225 genes were further analysed for pathway enrichment by IPA. Metabolic and cancer associated pathways were identified as the most significantly altered, further strengthening the suggested role for the p65 homodimer in controlling important aspects of metabolism and cancer (FIG. 2B). The top enriched pathways included oxidative phosphorylation (OXPHOS), sirtuin signalling pathway and mitochondrial dysfunction (FIG. 2B), which was further highlighted by the specific regulation of the genes involved in these pathways in p65^{-KO} cells as compared with CTR and the other NF-kB KO cells (FIG. 2C). The enrichment of metabolic pathways in p65^{-KO} AML cells as compared with CTR and the other NF-kB KO cells was mainly due to the downregulation of the genes involved in these pathways in p65^{-KO} cells (FIG. 2C). Importantly, the ChIP-ChIP NF-kB binding profile demonstrated specific binding of p65 within the genomic loci of these deregulated metabolic genes (FIG. 2D, lower panel, example of AKT1 and COX6A1 is shown). The p65 binding at these gene loci was inversely correlated to the RNA-seq read coverage (gene expression) in the p65^{-KO} U937 cells (FIG. 2C, 2D), suggesting p65-mediated regulation of these metabolic pathways in U937 AML cells.

The inventors of the present disclosure next sought to determine whether the predicted deregulation of metabolic pathways in p65^{-KO} cells could translate into demonstrable metabolic perturbation. To this end, the inventors of the present disclosure measured bioenergetic profiles of CTR and p65^{-KO} clones. Oxygen consumption rate (OCR)—an indicator of OXPHOS, and extracellular acidification rate (ECAR) for lactate production—an indicator of glycolysis, were measured using Mito- and Glycolysis-stress assays, respectively. When compared to CTR, the p65^{-KO} clones demonstrated an overall increased OCR and ECAR levels, suggesting deregulated metabolic function in the absence of p65 (FIG. 2E, 2G, data of all clones together). Importantly, all p65^{-KO} clones showed significantly increased basal OCR (p=0.03) relative to CTR clones, which was linked to increased proton leak, as no change in ATP synthesis was observed (FIG. 2F). p65^{-KO} cells also showed an increased maximal respiration (p=0.02), spare respiratory capacity (p=02, in response to FCCP stress) and non-mitochondrial respiration (p=0.03) (FIG. 2F). Analysis by glycolysis stress assays demonstrated an increased glycolysis and glycolytic capacity in the p65^{-KO} clones as compared to CTR clones, though no change in non-glycolytic acidification was observed (FIG. 2H). Upregulation of both mitochondrial respiration and glycolysis in p65^{-KO} clones suggest a potential “hybrid” metabolic state in the p65^{-KO} cells, that may be due to compromised cellular bioenergetics. This was supported by cell energy phenotype analysis (FIG. 2I). Taken together, the inventors of the present disclosure's data suggest that (i) p65 couples the gene regulation with metabolic pathways and (ii) p65^{-KO} cells are in “hybrid” metabolic state. This p65 metabolic plasticity could allow U937 cells to adapt to the hostile environments, which is often observed in cancers and could be also applied in the context of AML pathogenesis.

### Loss of p65 Aggravates Induced AML Pathologies

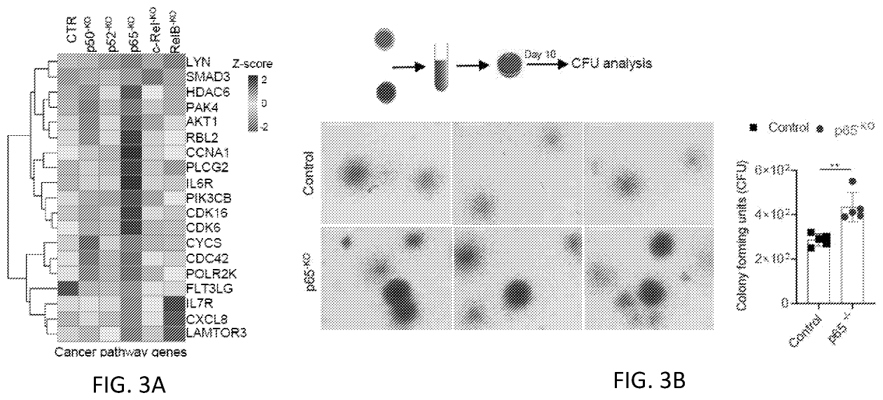

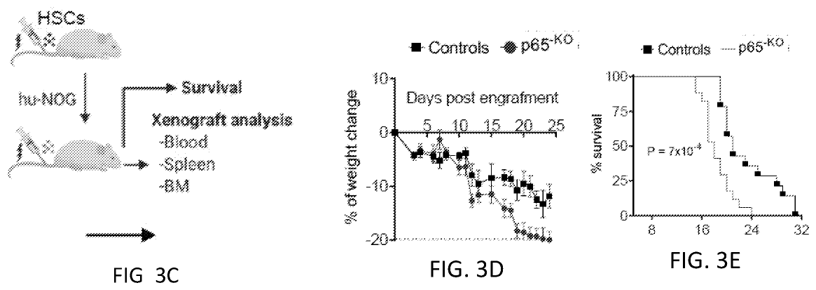

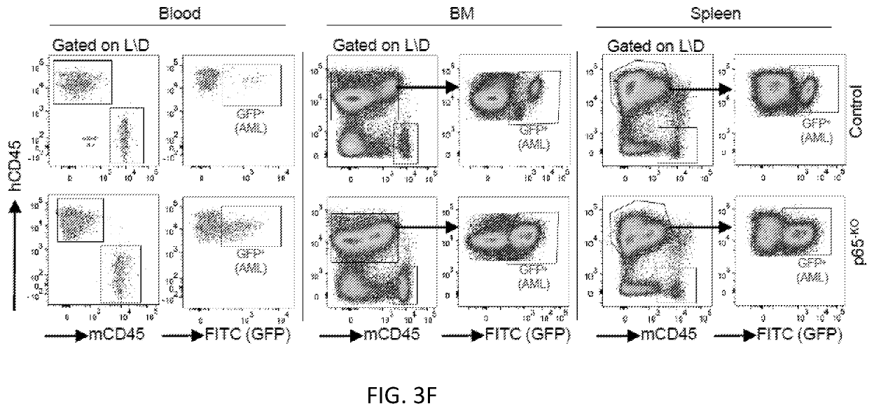

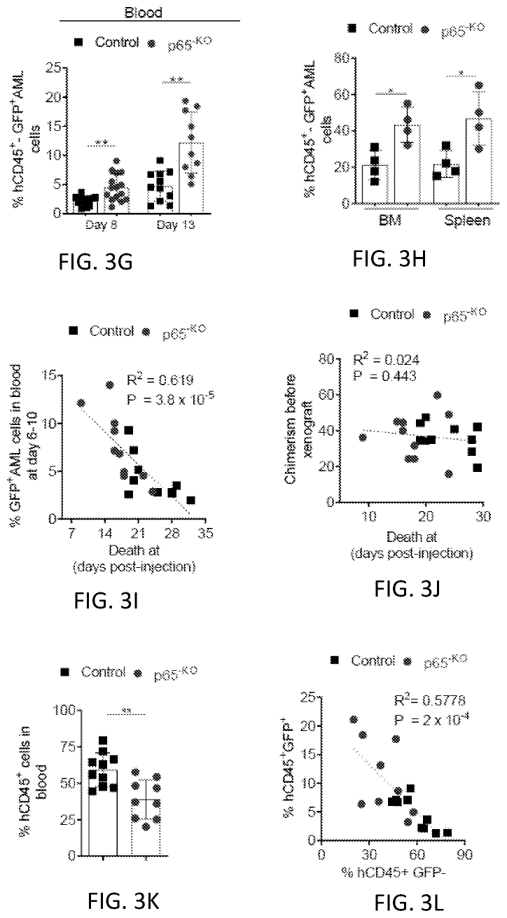

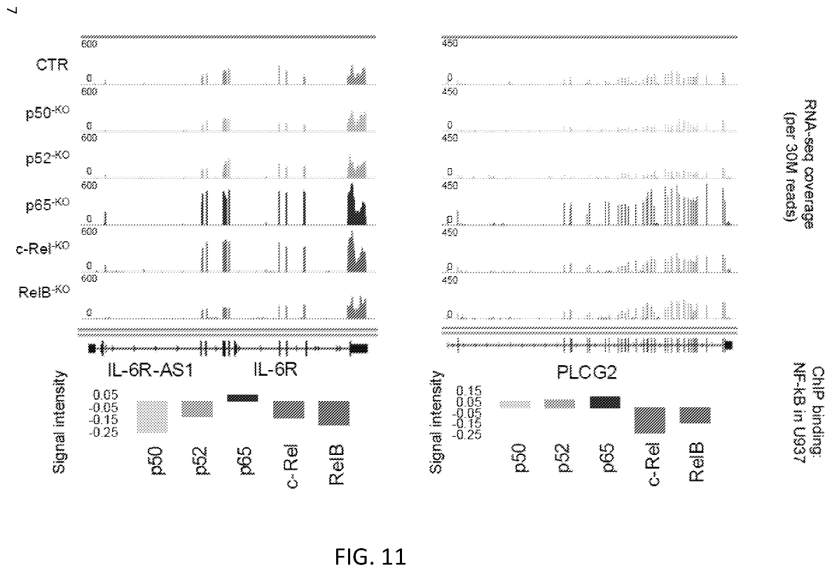

In addition to metabolic pathways, the p65 homodimer specific regulatory modules (225 DEGs)/DEGs specific to p65^{-KO} cells also showed an enrichment of cancer associated pathways including AML and AML associated signalling pathways; IL-6, IL-7, JAK, CXCR4, JAK-STAT and GM-CSF signalling (FIG. 2B). FIG. 3A to 3L shows p65 gene regulatory networks mediated suppression of AML progression. The genes associated with these AML-related pathways were highly deregulated in the p65^{-KO} clones as compared with CTR or the other NF-KB KO cells (FIG. 3A). As detailed above, the ChIP-ChIP NF-kB binding profile showed the specific binding of p65 within the genomic loci of these deregulated cancer-specific genes (FIG. 11). The p65 binding at these gene loci was directly correlated with the RNA-seq read coverage (gene expression) in the p65^{-KO} in U937 demonstrating an association of reduced p65 absence with increased gene expression. This suggests a potential inhibitory role of homodimer p65 in regulating leukaemia-specific pathways which in fact align with earlier reports showing its target specific role in the context of transcription regulation.

AML is characterized by the uncontrolled expansion and accumulation of leukaemia-stem cells (LSCs). Therefore, the inventors of the present disclosure sought to investigate whether the predicted deregulation of AML associated pathways in p65^{-KO} cells could translate into demonstrable LSCs functions, the inventors of the present disclosure therefore tested the effect of p65 deletion on LSC formation by methylcellulose colony-forming unit (CFU) assay which determine the functional capability of a cell to form LSC colonies. All p65 deficient clones (N=5 clones) found to have an enhanced ability to proliferate and form colonies as compared to CTR (N=5) clones (FIG. 3B), suggesting a negative role of p65 in LSC functions. Taken together, this data strongly links the p65-mediated energy metabolism plasticity with the stemness program in AML cells and indicate an anti-leukemic role of p65.

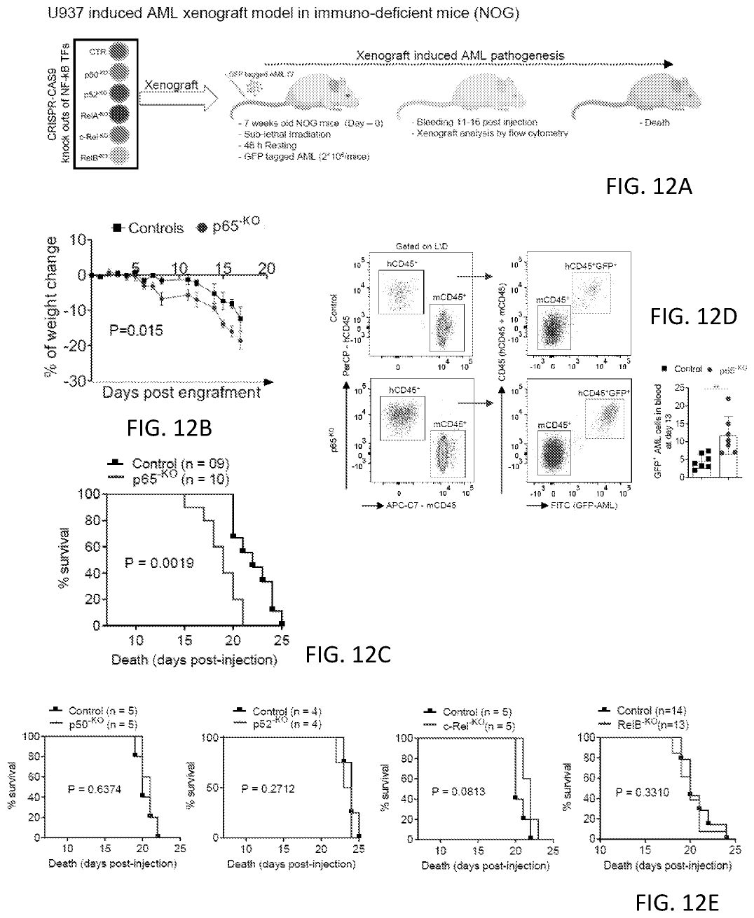

FIG. 12A to 12E show NFKB-p65 specific ant-leukemic role in induced AML xenograft model in immunodeficient NOD/shi-scid-IL-2Rganull (NOG) mice. To further investigate the potential “anti-leukemic” role of p65, the inventors of the present disclosure compared disease progression following injection of p65-KO and CTR unedited cells in a NOG xenograft mouse model (FIG. 12A). As shown in FIG. 12B, p65^{-KO} xenografts showed a severe weight loss in contrast to CTR xenografts suggesting a more aggressive progression of the p65-KO xenografts. The weight loss in the p65^{-KO} xenografts translated to their poor survival as compared to the CTR xenografts (FIG. 12C). Since the CRISPR-Cas9 edited clones used in this study are easily trackable due to constitutive expression of the fluorescent marker GFP, the inventors of the present disclosure analysed xenograft expansion by detection of hCD45⁺GFP⁺ in whole blood using flow cytometry (FIG. 12D, left panel). The hCD45⁺GFP⁺ AML cells in periphery were significantly greater in p65^{-KO}-injected NOGs as compared to CTR-injected NOGs (FIG. 12D right panel). The faster expansion of GFP+ AML cells in p65-KO-injected NOGs was also translated to the induced AML progression, i.e., p65-KO xenografts showed a severe weight loss and reduced survival (FIG. 12B). Furthermore, other p50^{-KO}, p52^{-KO}, c-Rel^{-KO}, and RelB^{-KO} were also analysed for their role in AML pathogenesis. As shown in FIG. 12E, no significant effect of their deficiency was observed in induced xenograft survival suggesting for a p65 specific role in AML pathogenesis.

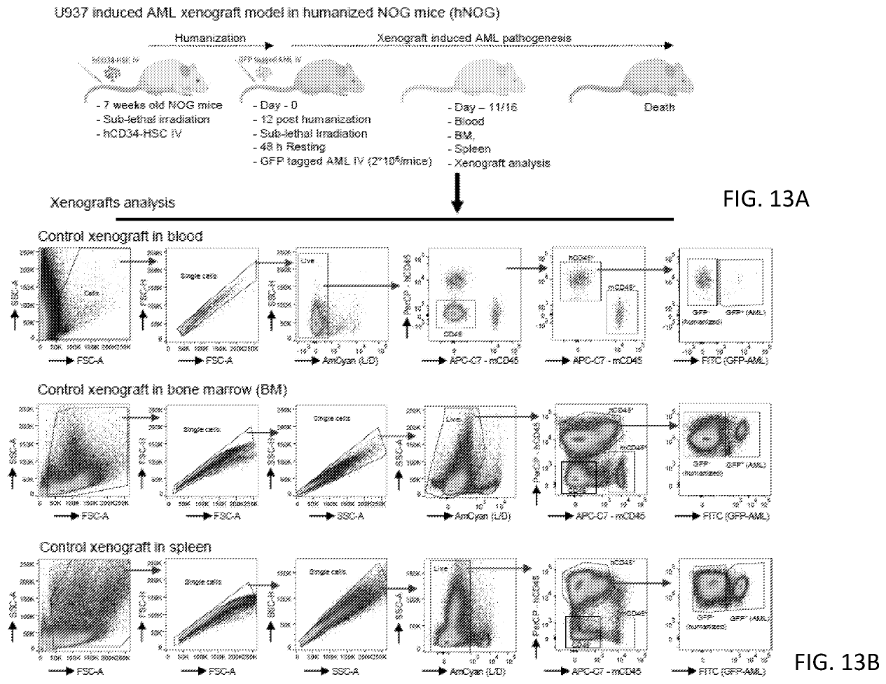

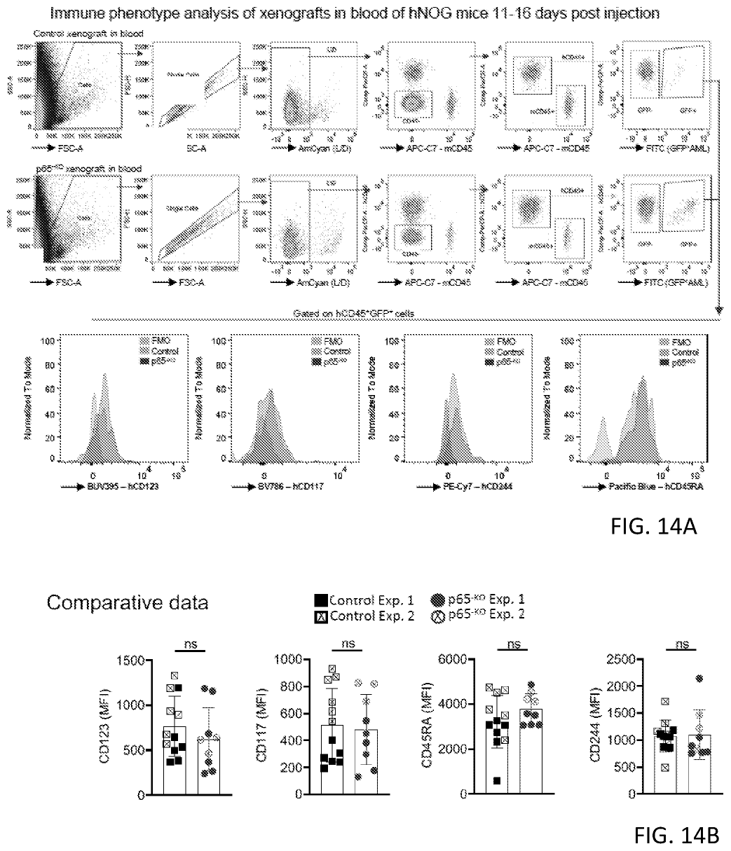

Haematological malignancies including AML are characterized by perturbed progenitor cells leading to impaired differentiation and enhanced proliferation, which ultimately leads to the bone marrow (BM) remodelling and dysfunctional normal haematopoiesis. To investigate this, the inventors of the present disclosure next analysed the engraftment of p65^{-KO} and CTR clones in an established humanized NOG (huNOG) xenograft model, which has a normal human-like haematopoiesis (FIG. 3C, FIG. 13A, 13B). FIG. 13A to 13B show U937 induced AML xenograft model in humanized immunodeficient (huNOG) mice. Consistent with NOG xenograft data (FIG. 12B, 12C, 12D), the p65-KO xenografts in the huNOG mice showed severe weight loss and poor overall survival as compared to CTR xenografts (FIG. 3C, 3D, 3E). The frequencies of hCD45⁺GFP⁺ cells in the blood appeared to be progressive in both p65^{-KO} and CTR huNOG xenograft groups but were significantly higher in the p65-KO hNOG xenograft (FIG. 3F, 3G see also FIG. 12D). Xenografts expansion/infiltration in tissues; BM and spleen were also analysed, similar to blood, we further showed an increased expansion of GFP+ AML cells in the BM and spleen of the p65^{-KO} xenografts compared to CTR xenografts (FIG. 3F, 3H). However, the inventors of the present disclosure could not find any differences in the expression of markers associated with AML pathogenesis; CD244, CD123, CD117 and CD45RA on blood hCD45⁺GFP⁺ cells between the p65^{-KO} and CTR xenografts (FIG. 14A, 14B). FIG. 14A to 14B show anti-leukemic functions of p65-KO in induced AML pathogenesis is independent of known LSC markers.

In huNOG mice the inventors of the present disclosure observed a significant inverse correlation between blood xenograft frequencies and survival viz. higher xenograft (observed in p65^{-KO} injected huNOGs) associated with poor survival (FIG. 3I). However, no significant correlation was observed when humanization before xenograft (Chimerism before injection of xenografts) was compared with survival (FIG. 3J). To further understand the impact of p65 deficiency on AML pathogenesis in the context of normal hematopoiesis, the inventors of the present disclosure analysed the frequency of human leukocytes (hCD45⁺, an indicator of hematopoiesis) in blood cells of huNOG mice. The inventors of the present disclosure noticed a significant decrease in the total hCD45⁺ frequencies in the blood of the p65^{-KO} xenografts as compared to CTR xenografts suggested an impaired normal hematopoiesis (FIG. 3K). Hence, a correlation analysis was performed between hCD45⁺GFP⁺ (xenograft) and hCD45⁺GFP⁻ (humanization derived). As shown in FIG. 3L, an inverse correlation was observed, which indicated induced AML disease progression significantly affecting the normal hematopoiesis. However, the impairment of normal hematopoiesis is very prominent in p65^{-KO} xenografts compared to controls, further suggesting an aggressive nature of p65^{-KO} AML cells (FIG. 3L). Taken together the data of the present disclosure suggest an anti-leukemic role of p65 where its reduction has an impact on normal hematopoiesis through competitive advantage of p65-KO AML cells, supporting a previous observation in the hematopoietic compartment of mice lacking RelA/p65.

The p65-KO Specific Deregulated Genes Constitute Novel Therapeutic Targets for AML Pathogenesis

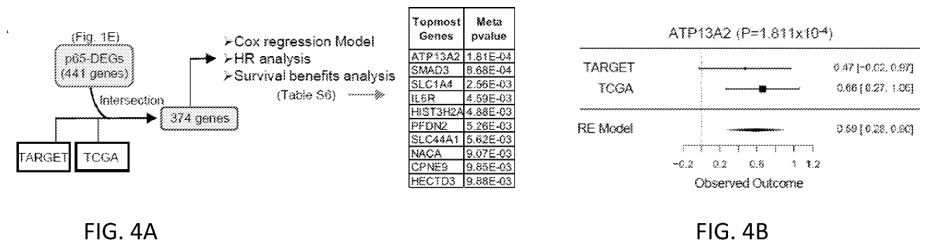

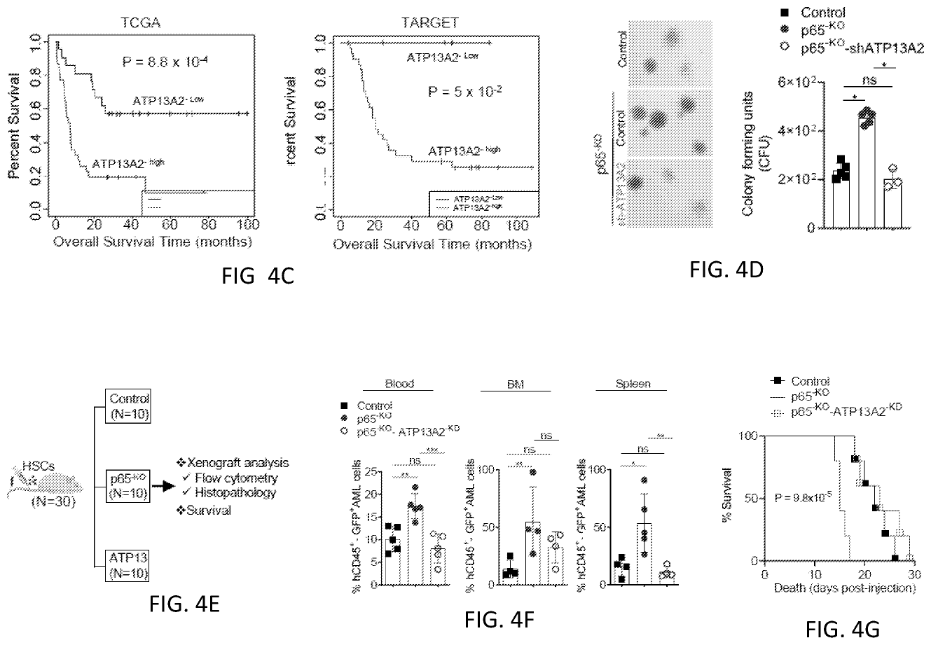

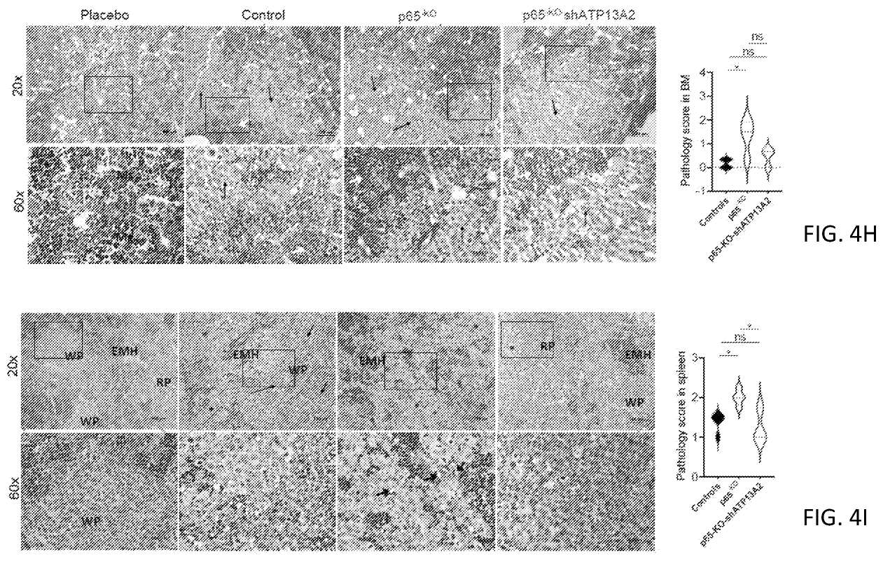

p65 deficient AML showed a perturbed gene signature leading to enhanced metabolic adaptation, LSC functions and xenografts expansion consequently poor survival in p65-KO induced AML xenografts, the inventors of the present disclosure next explored if the p65-dimer specific gene signature identified in U937 AML cells (441 genes, FIG. 1E) could be used as a prognostic predictor/to mine novel targets of clinical relevance in AML patients. FIG. 4A to 4I shows the identification of ATP13A2 as prognosis markers for AML using p65-KO gene signature as probe. Total 374 of 441 genes were consistently detected at mRNA level in two AML data sets analysed; TCGA (The Cancer Genome ATLAS) and TARGET (Therapeutically Applicable Research to Generate Effective Treatments) (FIG. 4A left panel F). Analysis of these 374 genes by a univariate Cox regression model identified 25 genes in TCGA (PDCL3, ATP13A2, ARL3, INPP5B, TNNT1, ZER1, SMAD3, CDKL5, VCL, GGCT, CPNE9, MGST1, VPS29, HIST3H2A, HERPUD2, TMEM60, CYCS, PPIL3, FHL2, AVL9, RIC8A, PSMA2, MTHFR, ADAMTS7, TIMM8A) and 32 genes in TARGET (RPSA, RPLPO, IL19, SLC1A4, RPL10, RPS2, RPL6, SKA2, RPS4X, VPS25, SLC44A1, BTF3, IL6R, TNNT1, HMOX1, PFDN2, RALGDS, RPL5, EXOC3, RAB13, IER3, YWHAB, GTSF1, RPL8, SLC25A26, GPT2, TCIRG1, TMEM14A, TIMM9, MSH5, ADA, SEC11C) datasets to be significantly associated with risk or protection from AML. Further meta-analysis that combined the results from the TCGA and TARGET identified 28 genes to be significantly associated with either risk, or protection in both TARGET and TCGA cohorts (FIG. 4A right panel). These genes are ATP13A2, SMAD3, SLC1A4, IL6R, HIST3H2A, PFDN2, SLC44A1, NACA, CPNE9, HECTD3, SLC30A5, RALGDS, CYCS, FHL2, PPIL3, CDKL5, GJB2, RPL6, RAB13, ADAMTS7, NDUFS4, HERPUD2, GLG1, MTHFR, PSMA2, TNNT1, TMEM14A, and MCTS1.

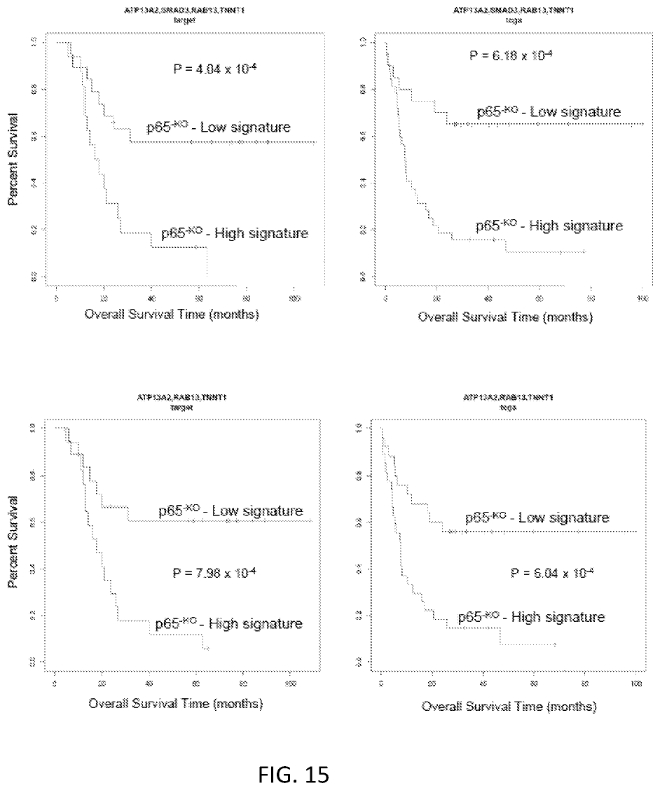

Forest plots summarizing HRs and CIs for top-most genes ATP13A2 also identified a significantly improved survival among AML patients in both datasets as shown in FIG. 4B, 4C. Furthermore, cox regression analysis was performed to identify combinations of robust 3-4 gene markers among the 28 identified candidates for the better prediction of overall survival (FIG. 15). Three and four gene signature model-based patient's stratification further improved prediction of overall survival in both AML cohorts (FIG. 15). Notably, ATP13A2 remained to be in the topmost gene signatures further suggesting for its critical molecular role in the p65^{-KO} associated aggressive AML pathologies. ATP13A2 codes for PARK9 protein a transmembrane lysosomal P5-type transport ATPase, which was upregulated in p65-KO AML cells (FIG. 4A). The expression of ATP13A2 was associated with survival among AML patients in the two datasets (hazard ratio 0.59, FIG. 4B).

To investigate the therapeutic relevance of ATP13A2, the inventors of the present disclosure used shRNA technique to suppress the expression of ATP13A2 in p65-KO AML cells (p65-KO-shATP13A2-1), comparable to the levels observed in CTR cells (FIG. 16A, 16B). These shRNA knock down and tested for LSCs functions using CFU assay (FIG. 4D). As shown in FIG. 4D, reduced expression of ATP13A2 in p65^{-KO} cells (p65^{-KO}-shATP13A2) leads to the loss of p65^{-KO} associated enhanced LSCs proliferation and colonies formation abilities.

The inventors of the present disclosure next analysed whether impaired ATP13A2 expression in p65^{-KO} also has an effect on p65^{-KO} associated aggressive AML pathogenesis in induced xenografts. To this end, in a cohort of 30 huNOG mice xenografts [control (n=10), p65^{-KO} (n=10) and p65^{-KO}-shATP13A2 (n=10)] were induced to analyse the disease progression (FIG. 4E). Xenograft expansions were analysed in 50% of mice at day 13 by flow cytometry in blood, BM and spleen. As expected, the xenograft expansion in p65^{-KO} group was significantly higher in blood, BM and spleen compared to control groups, however reduced ATP13A2 expression in p65^{-KO} cells completely reversed the p65^{-KO} aggressive expansion/infiltration in blood and spleen, whereas a moderate reversal observed in BM (FIG. 4F). In line with aggressive p65^{-KO} xenograft expansion in tissues compared to controls and p65^{-KO}-shATP13A2, p65^{-KO} xenograft showed poor survival which was reversed in p65^{-KO}-shATP13A2 xenografts (FIG. 4G). This further suggested ATP13A2 driven AML aggressiveness of p65^{-KO}.

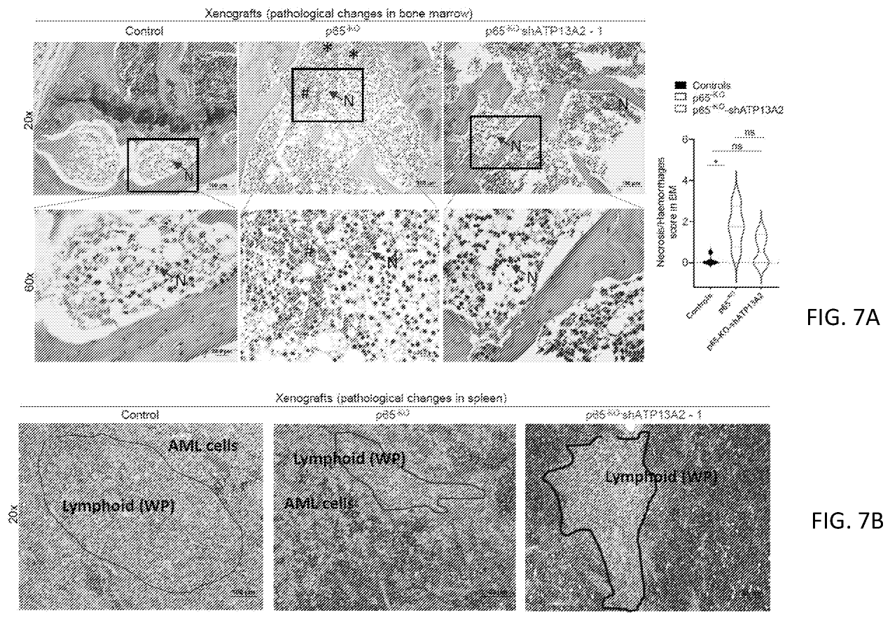

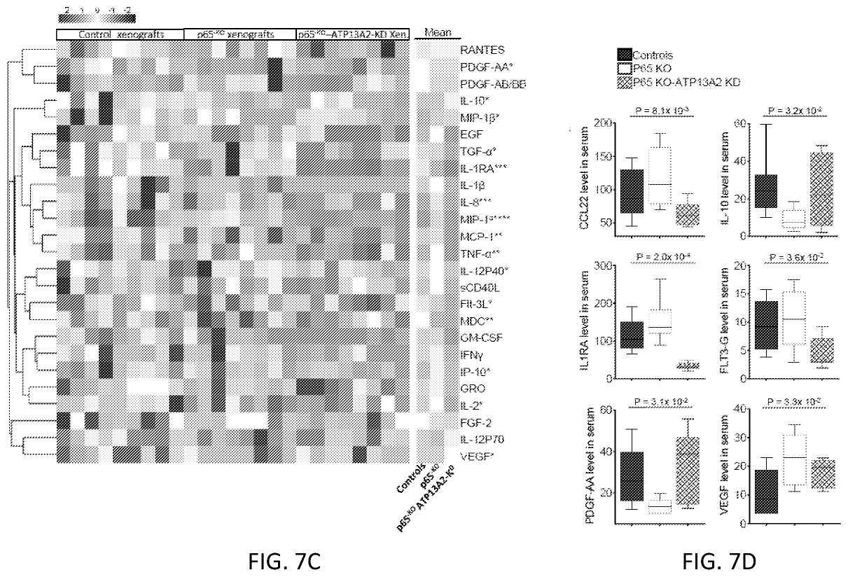

Further histological assessment using H&E revealed infiltration of AML cells in all three groups (control, p65^{-KO} and p65^{-KO}-shATP13A2), however infiltration appeared to be more prominent in p65^{-KO} group animals compared to Control and p65^{-KO}-shATP13A2 groups. Reducing expression of ATP13A2 in p65^{-KO} cells significantly impaired the infiltration of p65^{-KO}-shATP13A2 cells in spleen (FIG. 4I, left panel), while moderate reduction of p65^{-KO}-shATP13A2 xenografts infiltration in BM was observed (FIG. 4H, left panel). This data was consistent with flow cytometry analysis (FIG. 4F). The infiltration and expansion of AML cells also leads to the pathological changes. Therefore, a subjective scoring-based analysis (described in method section) was applied to analyse the pathological changes induced by controls, p65^{-KO} and p65^{-KO}-shATP13A2 xenografts. In general, infiltration of xenografts was predominant in p65^{-KO} xenografts than controls and p65^{-KO}-shATP13A2 in BM and spleen (FIG. 4H, 4I, right panel). Most prominent observed pathological changes were necrosis in BM and lymphoid depletion in spleen (FIG. 7A, 7B). Overall, pathological changes were more in p65^{-KO} xenografts than other two groups, which were reversed in p65^{-KO}-shATP13A2 xenografts. Taken together this suggests ATP13A2-dependent aggressiveness of p65-KO AML cells.

### ATP13A2 Mediates p65 Regulated Metabolic Adaptation of AML Cells

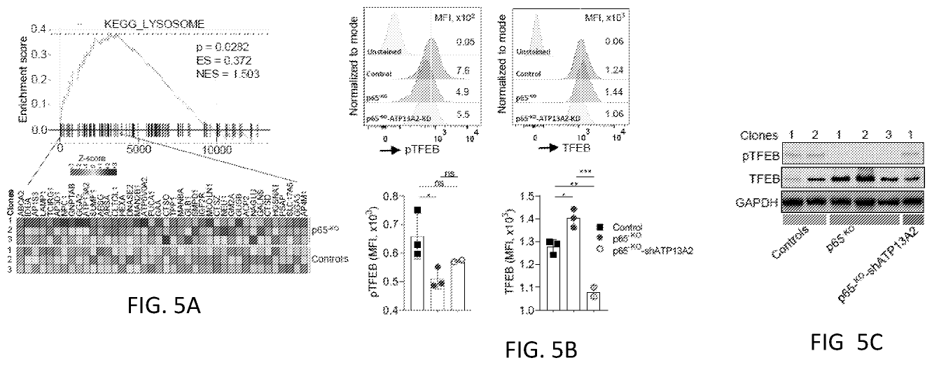

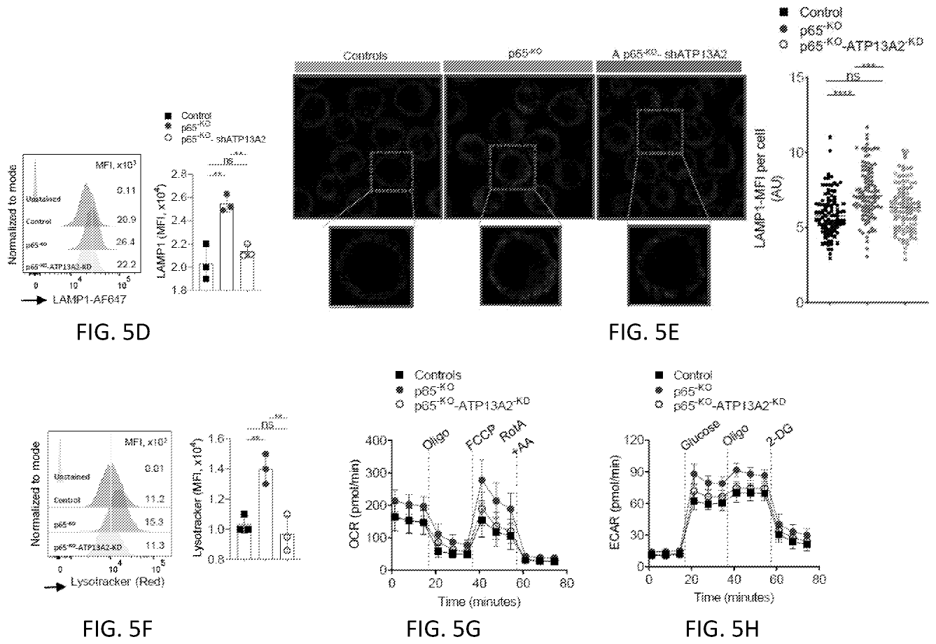

ATP13A2 mediates p65 associated metabolic adaptation. The ATP13A2 is a lysosomal P5-type transport ATPase (PARK9) that plays a critical role in lysosomal functions and also found to mediate the p65-KO associated AML pathologies. Therefore, the inventors of the present disclosure assessed the impact of downregulation of ATP13A2 expression in p65-KO cells on its lysosomal function. Similar to ATP13A2, the inventors of the present disclosure next analysed, if the loss of p65 in U937 AML cells also modulates other lysosomal genes/resulted in deregulation of the gene expression of lysosomal-associated proteins using gene signature enrichment analysis (GSEA). The GSEA of the expression data revealed elevated expression of a number of lysosomal genes in p65^{-KO}, which appeared to be mediated through a known downstream target of ATP13A2; TFEB (FIG. 5A, 5B, 5C). As shown in FIG. 5B left panel, intracellular staining flow cytometry analysis for the phosphorylation of TFEB at ser-211 which inhibit nuclear translocation and its target gene expression was decreased in p65^{-KO} cells, however total TFEB level found to be increase (FIG. 5B right panel). Consistent with flow cytometry analysis, western blotting further showed reduced ser-211 and vis-versa for total TFEB levels suggested a deregulation of TFEB associated functions in p65 deficient cells (FIG. 5C). Interestingly, the deregulation of TFEB in p65^{-KO} cells in fact mediated through ATP13A2 as shRNA knock down of ATP13A2 reversed the ser-211 phosphorylation and total TFEB levels in p65^{-KO} cells (FIG. 5B, 5C). This suggested a global deregulation of lysosomal function in p65-KO AML cells.

In order to further examine whether deregulated TFEB and upregulation in the lysosomal gene expression in p65^{-KO} cells also observed at cellular level, lysosomal membrane protein LAMP1 was analysed as an indicator of lysosomal mass by flow-cytometry and confocal microscopy. As shown in FIG. 5D, an increase in LAMP1 expression was observed in p65^{-KO} cells compared to controls. Also, a comprehensive and quantitative analysis of LAMP1 stained; control, p65^{-KO} and p65^{-KO}-shATP13A2 cells using confocal microscopy further demonstrated ATP13A2 dependent increased lysosomal mass in p65^{-KO} compared to controls (FIG. 5E). The ATP13A2 is also known to regulate lysosomal functions through acidification, therefore, cells were stained with a pH sensitive dye lysotracker to analyse the lysosomal acidification. P65^{-KO} cells having high level of ATP13A2 showed increased lysosomal acidification compared to control cells, which was reversed in p65-KO-shATP13A2 cells (FIG. 5F). In summary, this suggested a deregulated lysosome associated functions in p65^{-KO} cells which appeared to be mediated through ATP13A2 as reduced expression of ATP13A2 through shRNA knock down in p65^{-KO} cells reversed the deregulated lysosomal pathways; mass and acidification.

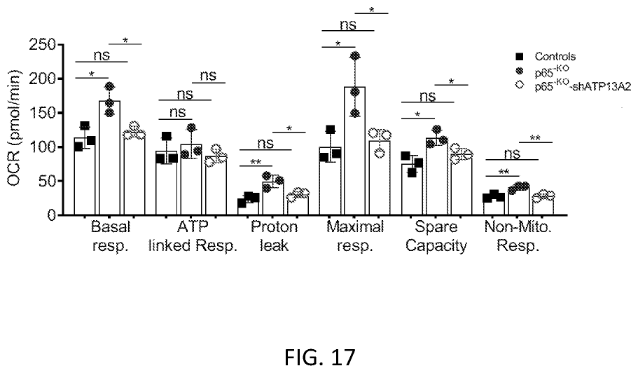

Lysosomal functions have been shown to influence cellular bioenergetics; therefore, the inventors of the present disclosure hypothesize ATP13A2 to be responsible for “hybrid” metabolic state of p65-KO cells (FIG. 2E to 2I). Therefore, the p65^{-KO} cells with reduced expression of ATP13A2 were analysed for their bioenergetics profiles using Mito- and Glycolysis-stress assays, respectively. As expected, p65^{-KO} cells appeared to have higher OCR and ECAR compared to controls (FIG. 5G to 5J). Analysis of overall mitochondrial OXPHOS and glycolytic functional parameters further suggested a dysfunctional mitochondrial functions, increased glycolytic functions and a hybrid bioenergetics state (FIG. 5G to 5J, FIG. 17). However, reduced expression of ATP13A2 in p65^{-KO} cells reversed the overall bioenergetics profiles (increased mitochondrial (OCR) and glycolytic (ECAR) phenotype) associated with p65^{-KO} cells (FIG. 5G to 5J, FIG. 17).

To further demonstrate the importance of targeting ATP13A2 in AML, we knocked down ATP13A2 in U937 cells by shRNA, and injected these cells in huNOG mice (FIG. 5K, left panel). U937 cells transformed with scrambled shRNA (control) were injected in huNOG mice. huNOG mice xenografted with shATP13A2 U937 AML cells survived longer (FIG. 5K, right panel).

Taken together, the data of the present invention links the aggressive nature of p65-KO cells with deregulated lysosomal and metabolic functions in these cells, and demonstrate the role of endolysosomal transporter ATP13A2 in that pathology. Furthermore, the present invention suggests for a novel p65-ATP13A2 axis that regulates metabolic adaptation through modulation of lysosome and bioenergetics pathways which defines pathophysiological nature of induced AML disease progression and also overall survival in human AML patients. The inventors of the present disclosure propose ATP13A2 as a novel potent target for AML therapy.

Aggressive p65-KO AML Cells Induce Pro-Leukemic cytokine/chemokine signature in xenografts in humanized mice

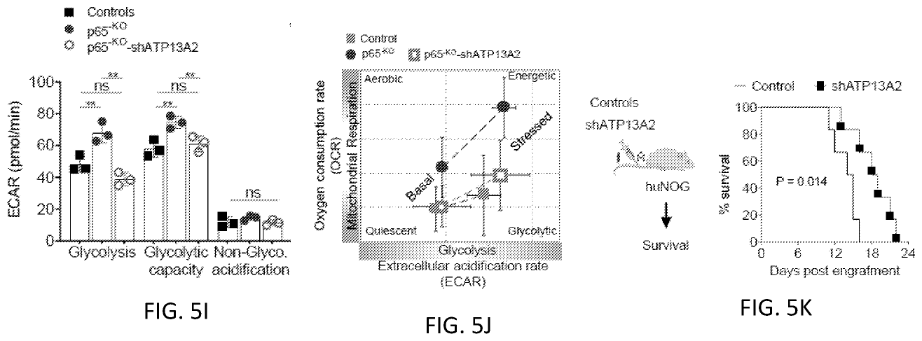

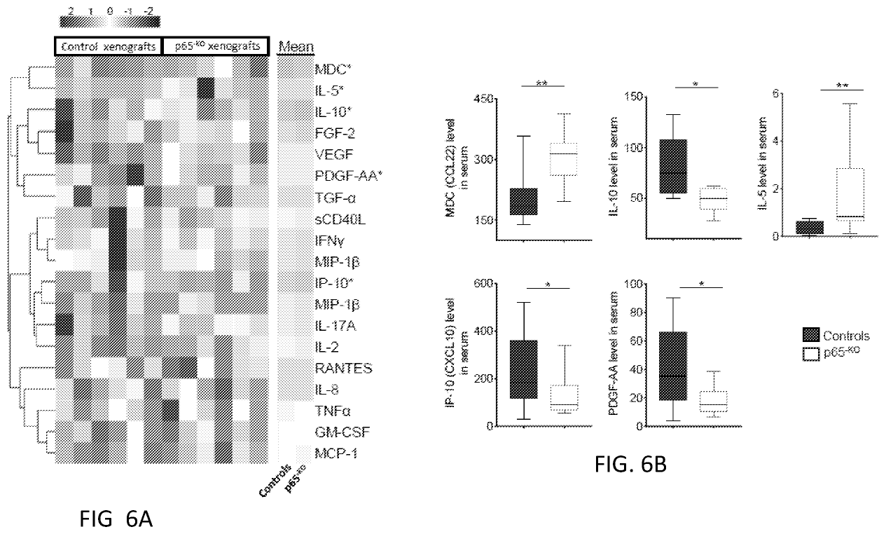

The inventors of the present disclosure used huNOG xenograft model and showed that blood/spleen/bone marrow (BM) of huNOG mice which received p65-KO xenograft had more AML cells (FIG. 3C to 3H). Since cytokines and chemokines play an essential role in the AML pathogenesis either through directly promoting the proliferation of AML cells or suppression of antitumor immune responses, the inventors of the present disclosure next analysed human cytokines and chemokines (using multiplex ELISA) in the serum from huNOG xenografts from FIG. 3C experiment. The inventors of the present disclosure found several human cytokines/chemokines such as MDC (CCL22), IL-10, IL-5, IP-10 and PDGF-AA to be significantly modulated in the serum of p65-KO xenografts compared to huNOG mice which received control xenografts (FIG. 6A). Notably, CCL22 level was increased, whereas the levels of IL-10 were decreased in p65-KO xenografts, which are known to be associated with antitumor functions of immune cells (FIG. 6B). Taken together this data suggest an anti-leukemic role of p65 where its reduction has an impact on normal haematopoiesis through competitive advantage of p65-KO AML cells and also induces pro-leukemic cytokine signature in xenografts.

Targeting ATP13A2 in Aggressive p65-KO AML Cells Reverses Lymphoid Depletion and Pro-Leukemic Cytokine/Chemokine Signature in Xenografts

The histopathological analysis of BM and spleen from huNOG mice demonstrated significant changes in overall pathologies in xenografts induced by p65-KO AML cells compared to controls (FIG. 4H, 4I). This was found to be mediated through ATP13A2 as reducing expression of ATP13A2 reversed the pathogenesis of p65-KO AML cells (FIG. 4H, 4I). Further analysis of the histopathology data revealed an extensive necrosis in the BM of p65-KO xenografts compared to controls (FIG. 7A). This necrosis was also associated with the depositions of amorphous granular eosinophilic materials, that is often accompanied with haemorrhages (FIG. 7A). In addition, the inventors of the present disclosure noticed a severe lymphoid depletion (immune suppression) in the spleen of p65-KO xenografts indicating a dysfunctional lymphoid immune compartment, that have been shown to play an important role in tumour surveillance and antitumor functions (FIG. 7B). Interestingly, the necrosis in BM and depletion of lymphoid cells in spleen were reversed when ATP13A2 expression was reduced in p65-KO AML cells (FIG. 7A, 7B). This data further supported a pro-leukemic role of ATP13A2, that involves not only leukemic stem cell functions, but also xenografts induced immune dysfunction.

The tumour induced immune suppression associates with aggressive disease progression and resistance to the therapy (chemo, immune and cellular therapies) and could be induced either by direct interaction or indirectly through deregulated immune regulatory cytokines/chemokines. This indeed also applies in the context of immune suppression (lymphoid depletion) observed in the xenografts induced by p65-KO AML cells (FIG. 7B). Therefore, the inventors of the present disclosure next determined human cytokines/chemokines to further understand the role of ATP13A2 on AML pathology. In general, huNOG mice which received ATP13A2-depleted p65KO AML cells, showed a reduced inflammatory state (FIG. 7C). Importantly, analytes, such as CCL22, IL10, IL1RA, PDGF-AA, FLT3-G and VEGF; that are known to be associated with AML pathologies and poor survival in AML patients; were found to be present in re3duced concentration in the serum of huNOG mice which received ATP13A2-depleted p65-KO AML cells compared to huNOG mice which received p65-KO AML cells (FIG. 7D). This reversal corroborated with the reversal in lymphoid depletion. Taken together, the data of the present disclosure links the aggressive nature of p65-KO cells with pro-leukemic cytokine and chemokine signature and immune suppression driven through ATP13A2.

Enhanced NF-kB activity has been frequently observed in cancers, which is largely viewed as an oncogenic property. However, opposing roles of NF-kB in cancers challenges this assumption. Likewise, perturbed NF-kB pathways is also reported in AML disease; nevertheless, their human specific pathophysiological functions are poorly understood. Here, the inventors of the present disclosure present a systematic and comprehensive cellular and pathophysiological functions of NF-kB TFs in AML pathogenesis using U937 as a AML cellular model. The inventors of the present disclosure first used a pgRNAs mediated CRISPR-Cas9 approach to knockout NF-kB TFs in U937 cells (FIG. 1A, FIG. 8). Global transcriptomic analysis revealed a selective impact of the deletion of NF-kB TFs on LPS induced inflammatory genes in U937 cells (FIG. 1C to 1E). Contrasting effects of the deletion of p65 and RelB on the gene expression in steady as well as inflammatory (LPS stimulation) state were observed (FIG. 1F to 1I). This might be due to the known inhibitory interaction of RelB and p65 as shown in previous studies known in the art.

Analysis of p65-dimer-specific target genes in unstimulated cells identified a dysregulation of metabolic signaling pathways in p65 deficient U937 AML cells, which correlated with higher energetic “hybrid” state, characterized by both increased ECAR and OCR (FIG. 2B, 2C, 2E to 2I). Metabolic programming and plasticity are hallmark of proliferative cancerous tissues. Interestingly, genes linked to AML and general cancer pathways were also found to be deregulated in the p65^{-KO} AML cells (FIG. 2B, 3A). AML disease originates from myeloid progenitor cells and is characterized by uncontrolled proliferation and expansion of LSCs which is enhanced in p65-KO U937 cells. This supports earlier reports showing expansion of progenitor cells in BM, enhanced stemness and tumor progression (FIG. 3B). Of note, stemness is linked to an upregulation of glycolysis, which is widely believed to be crucial for tumorigenesis. The inventors showed that the aggressive p65-deficient U937 AML cells had a “hybrid” metabolic state with an increased OCR and ECAR. This hybrid state may enhance the ability of cancer cells to satisfy the bioenergetic and biosynthetic needs for growth and may also influence the potential response of cancer cells to therapy. The aggressive blood, tissue expansion, pathological changes and poor survival induced by highly energetic p65-deficient U937 AML cells in immunodeficient, and humanized xenograft mice models further supports potential anti-leukemic functions of p65 and was shown to occur at the cost of normal hematopoiesis (FIG. 3C to 3L, FIG. 12).

To reveal the mechanism of pathologically aggressive p65^{-KO} AML cells, inventors used p65/p65 dimer specific differentially expressed genes to stratify the human AML patients in both TCGA and TARGET cohorts. The p65 specific gene signatures-based stratification and Univariate Cox regression analysis followed by Random effects model meta-analysis of the hazard ratios identified 28 genes significantly associated with either risk, or protection (FIG. 4A). The p65 dimer specific gene signature captured not only known prognosis and therapeutic markers (SLC1A4, IL6R, HECTD3, RALGDS, FHL2, MTHFR), but also uncharacterized markers such as ATP13A2 which was the top-ranked gene. The ATP13A2 showed an inverse correlation of its expression with overall survival (FIG. 4B, C), which indeed supports pathologically aggressive nature of p65^{-KO} AML cells (increase ATP13A2 expression) in vitro or induced xenograft models (FIG. 3C to 3L, FIG. 12). The increased expression and tumor promoting functions of ATP13A2 is reported in colon cancer and considered to be a novel prognostic/therapeutic biomarker. In line with inverse correlation of ATP13A2 with the overall survival of AML patients, impaired expression of ATP13A2 in p65^{-KO} cells reversed the pathological characteristics that further supports its AML promoting functions (FIG. 4D to 4I).

The p65 regulated ATP13A2 in U937 cells plays a critical role in TFEB mediated lysosomal homeostasis and functions. The enrichment of lysosome genes, increased TFEB activation in p65^{-KO} cells indicates a deregulated lysosome homeostasis and that was supported by an increased lysosomal mass and acidifications in p65^{-KO} cells (FIG. 5A, 5D, 5E). Deregulated lysosome homeostasis in p65 deficient AML cells was mediated through ATP13A2 which is consistent with earlier reports showing inhibition of TFEB activity and lysosome dysfunction in cells lacking functional APT13A2 gene in fibroblasts (FIG. 5B, 5C). Interestingly increase in lysosomal mass has been reported in AML compared with normal hematopoietic cells which align with our finding showing enhanced LSCs functions and aggressive nature of p65 deficient induced xenografts. Additionally, ATP13A2 also appeared to be the key to the energetic metabolic nature of p65^{-KO} cells as its reduced expression abrogated the increased glycolysis and mitochondrial respiration ultimately p65^{-KO} AML cells lost its highly energetic metabolic profile and also the pathological advantages (FIG. 5F to 5K). The finding in this present disclosure suggests for a novel p65-ATP13A2 axis that regulates metabolic adaptation through modulation of lysosome and bioenergetics pathways which defines pathophysiological nature of induced AML disease progression and also overall survival in human AML patients. Nonetheless highly bioenergetics metabolic state of p65 deficient AML cells not only makes them pathologically aggressive, but also more vulnerable to metabolic targeting agents which are currently being investigated or approved for the clinical use. The inventors of the present disclosure postulate that there could also be at least two AML patient endotypes based on p65-NF-kB activity that might help stratify the AML patients for their metabolic heterogeneity, diversity and dependency, thus reinforce the personalized treatment of AML patients and also merits further study to better understand the clinical implications of p65-ATP13A2 regulatory axis in AML pathogenesis and response to therapies.

Overall, the study of the present disclosure identified the state-specific homo- and heterogeneity among transcriptional regulation by the dimers of NF-kB family of TFs in a human AML cell line and established pathophysiological significance of the p65 dimer-dependent control of energy metabolism to metabolic adaptation in AML, and that defines a bioenergetics pathway controlled through ATP13A2 by which p65 can promote AML progression and survival. Also, p65 dimer specific gene signature captures known, and novel prognosis and therapeutic targets suggests its therapeutic implications, as p65 dimer-dependent gene signatures may help improve the exploitation of metabolic vulnerabilities for personalized therapy in AML.

### Materials and Methods

DNA Constructs and Paired Guide RNAs (pgRNAs) Cloning in Lentiviral Vector

The oligomer based pgRNA-CRISPR-Cas9 lentivirus approach to generate KOs is based on two sets of plasmids: paired gRNAs cloning vectors (pAdaptor and pDonor) and lentiviral expression vector (PHASE-DEST-CAS9-T2A-GFP) (FIG. 1A)

For the cloning of paired gRNAs into pDonor vector, a set of reverse complementary forward and reverse 25 bases long oligomers for each gRNA targeting NF-kB family of TFs were synthesized from IDT. As shown in FIG. 1, The pgRNAs were first cloned into the adaptor vector in pool using gRNAs specific reverse complementary forward and reverse primers (Table I), that involves equimolar mixing of forward and reverse primers, phosphorylation of at followed by annealing and ligation to the bbsl digested adaptor (adaptor+pairs of guide RNAs). Next, ligated pgRNAs to the adaptor were pooled and purified by AMPure beads at 1:1 ratio and further ligated to Bbsl digested pDonor followed by transformation in TOP10 cells and plated onto the Kanamycin selection plate (FIG. 1A). Five times more bacterial clones than paired guide RNAs were selected to confirm the representation of paired guide RNAs in pool cloning by Sanger sequencing. Target specific paired guide RNAs were identified based on Sanger sequencing. Guide RNAs were further shuttle efficiently to the DEST containing lentiviral CAS9 expression plasmid using LR-gateway reaction according to the experimental need (FIG. 1A).

### Lentivirus Production, Infection and Knock Outs

Viral particles were produced by co-transfection of lentiviral expression plasmids (containing paired gRNAs and wild type CAS9 derivatives) together with packaging plasmids (1:2:3 plasmid ratio) in Lenti-X cell line using Xfect polymer (1:0.5 ratio) in a 6 well plates. Viral supernatants were collected post 72 h post transfection and filtered using 0.45 μm filter followed by concentration using LentiX concentrator (Takarabio). Multiplicity-of-infection (MOI) of 10 was used to transduce the cells in the presence of 8 μg/ml of polybrene with a centrifugation at 2200 g for 1 h at 22° C. For single cell cloning to identify productive knock out clones, post 5-days of infection, cells were directly sorted in round bottom 96 well plates using constitutively expressed GFP. Single cell clones were expanded for two weeks, 90% expanded cells from 96 well plate was transferred in a new V shaped 96 well plate and pelleted by centrifugation followed by lysis of cell pellet in 50 mM NaOH at 96° C., for 30 min followed by NaOH neutralization by addition of 10% 1M Tris-Hcl (PH: 8.0). Plate was centrifuged at 4.5 K for 5 min and supernatant containing genomic DNA was collected to be used for the validation of genetic editing by PCR amplification. To validate the editing, targeted regions were amplified using specific sets of primers and sequenced. Based on Sanger sequencing, minimum two clones with editing for each target gene were selected to validate for the loss at protein level using western blotting (FIG. 1A and see also FIG. 8C).

### Western Blotting

Multiple selected potential KO clones for each factor were analysed for the loss of proteins due the CRISPR-Cas9 mediated editing (see also FIG. 8C. Protein lysates from 50,000 lysed cells in RIPA (50 mM Tris, 7.4, 1% NP-40 0.25%) Na-deoxycholate, 150 mM NaCl 1 mM EDTA supplemented with protease inhibitors) buffer were loaded onto each well of 10% 12 well PAGE and separated, transferred onto PVDF membrane. Membrane was blocked with 5% milk/BSA and proteins of were detected by factor specific antibody (NF-kB1 p105/p50 (D7H5M) Rabbit mAb #12540, NF-kB2 p100/p52 Antibody #4882, and RELB RelB (C1E4) Rabbit mAb #4922 from Cell signaling, c-Rel Antibody sc-71 (Santa Cruz biotechnology), NF-kB p65 Antibody (C-20): sc-372 (Santa Cruz biotechnology) and HRP-linked secondary (Anti-rabbit/mice IgG, #7074/7076 Cell Signaling).

### TLR Stimulation and Cytokine Detection

Cells were cultured at 37° C. (5% CO₂) in RPMI supplemented with 10% FCS, L-glutamine (2 mM), penicillin (100U/ml), streptomycin (100 mg/ml) and Na-Pyruvate. On the day of stimulation, cells were seeded in 12 well plates (1×106/ml) in two sets and allowed 2 h rest. After 2 h, cells were stimulated with 0.5 μg/ml LPS (InvivoGen; LPS-EB (LPS from E. coli O111: B4). Following 6 h post-stimulation, cells in the first set were lysed in 1 ml of trizol/well for RNA isolation and second set of stimulation was continued for 24h, supernatants were collected and LPS induced cytokines (IL-6 and IL1-β) were analysed using Ready-SET-Go Elisa Kit (eBioscience).

### RNA Extraction and RNA Sequencing

Total RNA was extracted from bulk cells in two steps, RNA isolation by acid guanidinium thiocyanate-phenol-chloroform extraction (TRIzol, Thermo Fisher Scientific, Waltham, MA, USA) and the aqueous phase was processed using Qiagen RNeasy Micro clean-up procedure (Qiagen, Hilden, Germany). Purified RNA samples were quality checked using Agilent RNA 6000 Pico assay (Agilent Technologies, Paolo Alto, CA). Reverse transcription and amplification were performed according to established method. Briefly, cDNA was synthesized from 2 ng of purified total RNA using modified oligo (dT) primers along with 1 μl of a 1:50,000 dilutions of ERCC RNA Spike in control (Ambion® Thermo Fisher Scientific). To generate sufficient quantities of cDNA for downstream library preparation steps, 13 cycles of PCR amplification was performed. The quantity and integrity of cDNA was assessed using DNA High Sensitivity Reagent Kit, Perkin Elmer LabChip GX (PerkinElmer, Waltham, MA, USA). Subsequently, pooled cDNA libraries were prepared (250 pg of cDNA per sample) using Nextera XT Kit (Illumina, San Diego, CA, USA) with dual indices for de-multiplexing. The libraries were qPCR quantified (Kapa Biosystems, Wilmington, MA) to ascertain the loading concentration. Samples were subjected to an indexed PE sequencing run of 2×151 cycles on an Illumina HiSeq 4000.

### RNA Sequencing Data Analysis

Reads obtained from Illumina sequencing were assessed for quality using FASTQC version 0.11.7. Salmon version 0.11.3 was used for quasi-mapping-based quantification of transcript abundances from the paired end reads where the reference set of human transcripts was obtained from Gencode version 29. Transcript-level counts generated by Salmon were summarized to gene-level counts using tximport R/Bioconductor package version 1.2.3. The gene counts were imported into DESeq2 for the analysis of differentially expressed genes (DEGs). A negative binomial generalized linear model (GLM) was fitted to the counts data which included coefficients to model the effect of tissue type (CTR/c-Rel-KO/p50-KO/p52-KO/p65-KO/RelB-KO), treatment (no treatment/LPS) and sample batch (one of the three batches of experiments in which the samples were processed). Size factors and dispersion parameters in the GLM were estimated from counts data. The counts were adjusted by the median ratio method to normalize for library sizes. A Wald test was performed on the model coefficients to identify DEGs. The DEGs were selected fulfilling three criteria: nominal p-value <0.005, at least 1.5-fold change, and a baseMean (average counts across all samples after library size normalization) of more than 10. Biological pathways and functions enriched in the DEGs were analyzed using Ingenuity Pathway Analysis® (IPA). Custom scripts were written in R to perform principal component analysis (PCA) and to analyze correlations between samples. In these analyses normalized gene abundances were expressed as log transformed transcripts per million mapped reads, log 2 (TPM+1.0), where a pseudocount of 1.0 was added to prevent negative values, and only genes with baseMean >10 and having an Entrez gene id and symbol (n=12,845 genes) were used. The R package FactoMineR was used for PCA and pheatmap was used for drawing heatmaps. Sample correlations were evaluated by Spearman's correlation using the cor.test function of the R base library.

### Analysis of ChIP-ChIP Data

To analyse the binding of NF-kB transcription factors to the promoters of differentially expressed genes, data from a previous NF-kB ChIP-ChIP experiment on U937 cells was obtained from ArrayExpress with the accession ID E-WMIT-6. Raw two-color microarray data was processed using the marray Bioconductor package in R to perform background correction and within array normalization by the loess method. The resultant signal intensities were quantile normalized across all arrays in the dataset. Final ChIP signal intensities were calculated as average of the triplicates.

### Bioenergetic Assays

Metabolic assessment through extracellular flux analysis: analysis of the oxygen consumption rate (OCR) and the extracellular acidification rate (ECAR). Utility plate (Research Instrument) was hydrated with sterile water overnight in 37° C., non-CO₂ incubator. 50 ml falcon tube containing 20 ml of calibration buffer was also incubated overnight in 37° C., non-CO₂ incubator. Next day, U937 controls and p65^{-KO} cells (1×106/ml in complete medium) were seeded in each well of 12 well plate for 6 h, following 6 h of incubation, OCR and ECAR were measured using an XFp analyzer (Agilent Seahorse Technologies). XFp Seahorse plates were seeded with 200,000 U937 control and p65-KO cells per well of poly-L-lysine coated Cell culture plate (Sigma). Culture medium was replaced by XF base medium (Agilent Seahorse Technologies) supplemented with 2 mM glutamate, 2 mM sodium pyruvate and 11 mM glucose with an adjusted pH of 7.4. Cells were then incubated at 37° C. and 5% CO₂ for one hour.

Four compounds from the XFp cell mito stress test kit (Agilent Technologies) was injected during the assay at the following final concentrations: oligomycin (1.5 μM), ptrifluoremethoxyphenylhydrazone (FCCP, 0.5 μM), and a mixture of antimycin A (0.5 μM) and rotenone (0.5 μM). The increase in OCR after application of FCCP reflects the spare respiratory capacity (SRC) of a cell, the maximum respiratory rate that can be reached. ECAR rate was verified through application of the glycolysis stress test kit (Agilent Seahorse Technologies). For the glycolysis stress test kit assay medium was just supplemented with 2 mM glutamate. After manual injection of 10 mM glucose, 1 μM oligomycin was added, followed by 50 mM 2-deoxyglucose. The injection of glucose to the so far glucose-free medium allowed for correlation of an increase in ECAR with a higher glycolytic rate. After application of oligomycin the glycolytic capacity of a cell can be calculated, which is the maximum glycolytic rate that can be achieved. Agilent Seahorse Software Wave 2.3 was used for data analysis. The seahorse XF cell energy phenotype test report was generated through wave 2.3 report generator using assay result data from the seahorse XF cell mito stress test.

### Methylcellulose Human Colony-Forming Unit (CFU) Assay

The day before cells plating for CFU, MethoCult optimum medium-without EPO (STEMCELLS technologies) was transferred to 2-8° C. from −20° C. U937 controls and p65^{-/-} cells (1×10⁶/ml in complete medium) were seeded in each well of 12 well plate for 6 h. Following six h of incubation, cells were either washed with IMDM with 25 mM HEPES (wash medium) and were resuspended at 5×103/ml cells to make 10× cell suspension for the untreated cells or stimulated with 20 ng/ml of IL-6 for 12 h followed by washing with IMDM with 25 mM HEPES (wash medium) and were resuspended to make 10× cell suspension. Thawed MethoCult medium was shaken vigorously for 1-2 minutes and then let stand for at least 5 minutes, until all bubbles rise to the top, before aliquoting. Aliquots were prepared MethoCult™ medium into 14 mL using 16 gauge blunt-end needle. 100 μl (500 or 1000 cells) of cell suspension/plate were added directly to pre-aliquoted tubes of complete MethoCult™ medium and mixed gently to make a homogeneous cell suspension. 1.1 ml of cell suspension in MethoCult was plated to 35 mm dish in triplicate for each KO clone and incubated at 37° C., in 5% CO₂ with ≥95% humidity for 10 days. Colonies were scanned using EVOS M7000 (Thermo Fisher Scientific) and counted manually.

shRNA Knockdown

Knockdown of ATP13A2 in U937 and p65^{-KO} cells were performed using pLKO based shRNA strategy (FIG. 17). transduced using lentiviral shRNA pLKO vector. Transduced cells were selected with 1 g/ml puromycin for 10 days. The shRNA knockdown efficiency was validated using qPCR RNA isolation and quantitative RT-PCR (qRT-PCR). Total mRNA from cells was isolated using the RNeasy Mini Kit (Qiagen) and reverse transcribed (BIO-RAD; #1708891) according to the instruction in the manual. A real time PCR was performed with 10 ng of cDNA and oligonucleotide primers (300 nmol/L) in AB Biosystem 7000 (primer sequences are given in Table 2). The following PCR conditions were used for Light Cycler: 2 min, 50° C., and 10 min, 95° C., followed by 40 cycles of 15 s, 95° C. and 1 min, 60° C. in 15 μl reactions.

TABLE 2
Primers used throughout study

| | | | | | | PCR |
|---|---|---|---|---|---|---|
| | Targeted | Forward Primer | Reverse Primer | | Opt. | Master |
| Gene | Exon | (5′-3′) | (5′-3′) | Amplicon | Tm | used |
| NFKB1 | Exon | TGGCGAGCTTAA | AACAATATGTGTG | 502 | 60.5 | 2xLong |
| (p105/ | 1 | CACGAGG (SEQ | AAAGCGGGTG | | | Amp |
| p50) | | ID NO: 21) | (SEQ ID NO: 22) | | | master |
| | | | | | | mix |
| NFKB1 | Exon | TGGCAGCAGCAA | GGGTACTTTCAG | 1031 | 61.5 | 2xLong |
| (p105/ | 4 | TTTAAGACAAG | GCTCTCTATGG | | | Amp |
| p50) | | (SEQ ID NO: 23) | (SEQ ID NO: 24) | | | master |
| | | | | | | mix |
| NFKB2 | Exon | ATCTCGCTCTCCA | CACCTCCCATCT | 455 | 60.5 | 2xLont |
| (p100/ | 1 | CCGGATC (SEQ ID | CCAAGTCTTC | | | Amp |
| p52) | | NO: 25) | (SEQ ID NO: 26) | | | master |
| | | | | | | mix |
| RELA | Exon | GGGAGGATGCTG | AAGTGAGTAATC | 564 | 64 | 2xLong |
| (p65) | 1 | AGTCAAGG (SEQ | GGCGGACC (SEQ | | | Amp |
| | | ID NO: 27) | ID NO: 28) | | | master |
| | | | | | | mix |
| | | | | | | + |
| | | | | | | DMSO |
| REL | Exon | TGACCCGGGGTG | TTCGTCCTCCTTT | 504 | 60.5 | 2xLong |
| (c- | 1 | CAAGAATTC (SEQ | GGTTCGCTTATG | | | Amp |
| REL) | | ID NO: 29) | (SEQ ID NO: 30) | | | master |
| | | | | | | mix |
| REL | Exon | GCCTCTCCCAGC | TGTGGAGATGAC | 411 | 61.5 | Phusion- |
| (c- | 2 | CAATCTC (SEQ ID | TGTGAAGAAAC | | | Thermo- |
| REL) | | NO: 31) | (SEQ ID NO: 32) | | | scientific |
| | | | | | | GC |
| | | | | | | buffer |
| RELB | Exon | CTCAGTTGTCTC | CTCATCACCCTCT | 636 | 63 | KAPA |
| | 1 | GTCCAGAGC | GCCTTCTTC (SEQ | | | Biosystem |
| | | (SEQ ID NO: 33) | ID NO: 34) | | | GC |
| | | | | | | buffer |
| | | | | | | + |
| | | | | | | DMSO |
| RELB | Exon | TAGCTGGGATTA | CACTCGTAGCGG | 619 | 63.5 | Phusion- |
| | 4 | CAGACGTGGGAG | AAGCGCA (SEQ | | | Thermo- |
| | | (SEQ ID NO: 35) | ID NO: 36) | | | scientific |
| | | | | | | GC |
| | | | | | | buffer |
| | | | | | | + |
| | | | | | | DMSO |
| Adaptor_ | NA | TGTAAAACGACG | GCTATGACCATG | 593 | 61.5 | 2xLong |
| Uni | | GCCAGTGC (SEQ | ATTACGCCACG | | | Amp |
| | | ID NO: 37) | (SEQ ID NO: 38) | | | master |
| | | | | | | mix |
| qPCR primers | | | | | | |
| ATP1 | | GTTATCCAGGCT | GTGGACGATGAT | | | |
| 3A2 | | CTGCGAAGGA | CAGATGCTCC | | | |
| | | (SEQ ID NO: 39) | (SEQ ID NO: 40) | | | |

### Immunofluorescence Staining and Analysis

Cells were collected in 96-well plates for staining. Briefly, cells were washed once in PBS then fixed in 4% PFA for 15 min at RT. Cells were permeabilized with 0.1% Triton X-100 for 10 min and washed with PBS followed by blocking in 3% BSA 0.1% Triton X-100 for 1 h. After blocking, cells were washed with PBS and further stained with antibodies in blocking buffer with specific antibody dilutions (LAMP1 (1:250), TFEB (1:250), pTFEB (1:250)) for overnight with slow rotation. Cells were washed thrice in PBS and incubated in Anti-Rabbit AF647 (1:500) or anti-mouse-AF594 (1:500) diluted in blocking buffer at RT for 1 h. Cells were washed thrice and stained with Hoescht dye (1:1000) in PBS for 15 min. Cells were washed and loaded onto Poly-L-lysine coated Ibidi chambers for visualization with Olympus Confocal system. Mean fluorescence intensity of LAMP1 was calculated using the ImageJ software by manual selection of 100 cells per group (Fields per group=3)

### AML Xenograft Study

All AML xenograft experiments were performed as outlined in FIG. 12A and FIG. 13A. NOG mice (4-6-week-old) were sub lethally X-irradiated (1.2 Gy) two days before transplantation. GFP+ CTR and p65-KO U937 cells were injected into the mice via tail vein (2×10⁶ cells per mouse) in a final volume of 100 μL of PBS. The humanized NOG (huNOG) mice were generated by sub lethal X-irradiation (1.2 Gy) of 7-8 weeks old NOG mice followed by tail vain injection of 50,000-60,000 CD34⁺ human cord blood derived HSCs/mouse. Successful engraftment was confirmed by flow cytometric analysis of hCD45⁺ vs mCD45⁺ cells in the peripheral blood at 4, 8 and 12 wks post human HSC transplant. At 12-14 weeks post human HSC transplant, mice were sub-lethally irradiated and after two days U937 cells were injected as described for the NOG xenograft. Mice weight was recorded daily. On days 5-7 and 11-16 post-AML cells injection, peripheral blood was collected from the retro orbital sinus using heparinized capillary tubes (MARIENFELD) for xenograft analysis. At endpoint, the inventors of the present disclosure collected BM. The phenotyping of the xenografts in blood and BM was performed as described in the flow cytometry method section.

### Histopathology

Mice were euthanized and bone marrow & spleen tissues were harvested and fixed in 10% neutral buffered formalin (Sigma Aldrich, #HT501128) at room temperature for more than 48 hours. Femur bone tissues were then decalcified in Osteo Soft (Merck, USA), trimmed and processed routinely for histologic evaluation. The sections were cut at 5 mm thickness and stained with H&E. Tissues were then viewed under an Olympus BX53 upright microscope and pictures were taken with an Olympus DP71 digital camera using an Olympus DP controller and DP manager software (Olympus Life Science, Japan). Pathology evaluation was carried out following the subjective scoring such as: No lesions: 0, Minimal: 1, Mild: 2, Moderate: 3, Marked: 4, Severe: 5.

### Flow Cytometry

Anti-human antibodies for flow cytometry used in the experiments were: hCD45-PerCP-Cy5.5 (Thermo Fisher Scientific); hCD44-BV650, hCD117-BV785, hCD33-APC, hCD244-PE-CY7 (eBioscience); mCD45-APC-Cy7, hCD45RA-EF450 (BioLegend); hCD34-PE, hCD123-BUV395 (BD Bioscience). Whole blood staining was done by RBC lysis cells using ACK buffer (1×RBC lysis buffer, Thermo Fisher Scientific) for 5 min at RT followed by washing with PBS. RBC lysed blood cells were stained with live-dead (L\D) dye (UV 1:500, Thermo Fisher Scientific) for 30 min. Post LID staining cells were washed once with FACS buffer (PBS, 2% FCS, 1 mM EDTA. 0.1% sodium azide) and further stained with the antibody cocktail in FACS buffer with specific antibody dilutions [hCD45-PerCP-Cy5.5 (1:50), hCD44-BV650 (1:40), hCD33-APC (1:10), hCD117-BV785 (1:20), hCD244-PE-CY7 (1:20), mCD45-APC-CY7 (1:100), hCD45RA-EF450 (1:30), hCD34-PE, (1:10), hCD123-BUV295 (1:25)] for 15 min at 400° C. protected from light. Cells were washed once with FACS buffer and acquired using LSR II-5 Laser (BD Bioscience). Flow cytometry data were analysed with through FlowJo (version 10.4, TriStar). The overall gating strategies are outlined in FIG. 13B to FIG. 14A.

p65-p65 Dimer Regulated Gene Survival Analysis in AML Datasets

Gene expression and survival data was obtained from cBioPortal for the AML datasets TARGET and TCGA. Only patients went through standard chemotherapy without BM transplantation were selected, additionally PML-RAR samples were also excluded from the analysis due to its interaction with p65. Univariate Cox regression analysis was done for the p65 regulated 374 (out of 441) genes in each of the two datasets. Random effects model meta-analysis of the hazard ratios was then conducted to obtain a combined hazards ratio depicted as forest plots. The meta-analysis revealed a list of 29 genes from which panels of all possible 3, 4 genes were constructed. Each of these panels were then used in multivariate Cox regression analysis for each of the three datasets to determine the predictive performance of the panels as indicated by its likelihood ratio test P value. A rank sum approach was then used to rank the panels using the results from the three datasets and a combined P value computed using the Fisher's method. Analyses were done in R version 3.6.2 using the survival and metafor packages for Cox regression analysis and meta-analysis of the hazard ratios respectively.

### Statistical Analysis

All values are mean±SD or SEM of individual samples. Data analysis was performed with either R version 3.6.2 or GraphPad Prism Software (GraphPad Software Inc., version 7.01). The statistical tests utilized have been indicated in respective sections and figure legends.

### Accession Codes

GEO accession of RNAseq data generated in the study of the present disclosure is GSE153158.

### DETAILED DESCRIPTION OF FIGURES

Example embodiments of the disclosure will be better understood and readily apparent to one of ordinary skill in the art from the following discussions and if applicable, in conjunction with the figures. Example embodiments are not necessarily mutually exclusive as some may be combined with one or more embodiments to form new exemplary embodiments. The example embodiments should not be construed as limiting the scope of the disclosure.

FIG. 1A shows a diagram with an overview of the generation of p50, p52, p65, c-Rel and RelB KO in U937 cell line using oligomer based pgRNA approach (see also FIGS. 8 and 18A).

FIG. 1B shows a heatmap of the functional validation of KO cells upon LPS stimulation. Analysis of induced IL-6 and IL-1β secretion in culture supernatant. Data from one experiment out of two experiments, performed in triplicate is shown. The heatmap depicts the functional consequences due to the loss of NF-kB TFs; p50, p52, p65 c-Rel and RelB in AML cells. AML U937 cells were evaluated for the potential loss of cellular function through analysis of LPS mediated induction of inflammatory proteins. Upon LPS stimulation, impairment in the induction of IL-6 and IL-1β proteins in all KO clones of p50, p52, p65 and c-Rel was found, however no effects were observed for RelB-KO clones as compared to unedited control cells (CTRL). Overall, this shows that the NF-kB TF associated cellular functions i.e., inflammatory response to LPS, which is known to be mediated through NF-kB TFs was lost. This defect was due to the loss of targeted proteins among NF-kB TF KO clones (FIG. 8C).

FIG. 1C shows in the left panel, a diagrammatic representation of RNA-seq analysis on NF-kB TFs KO cells, and in the right panel a PCA analysis on 12845 genes (see also FIG. 9). UT—untreated cells, LPS—LPS treated cells, CTR—Control unedited cells.

FIG. 1D shows a Venn diagram showing the number of DEGs observed in LPS stimulated vs unstimulated NF-kB KOs and CTR cells.

FIG. 1E shows a Venn diagram showing the number of DEGs observed in unstimulated cells, NF-kB Kos vs CTR.

FIG. 1F shows a heatmap of the Spearman's ranked correlation between independent clones of factors specific NF-kB KOs based on the total set of LPS associated DEGs. Spearman's correlation analysis based on LPS associated 430 DEGs showed homogeneity among independent KO clones as their correlation were highest and also identified two major functional aspects of NF-kB represented by canonical cluster (mid-bottom right, p65^{-KO}, p50^{-KO}, c-Rel^{-KO} and p52^{-KO} clones) and non-canonical cluster (top left, RelB and CTR clones). Overall, this shows that the canonical NF-kB TFs are the major driver of LPS induced inflammatory response compared to non-canonical NF-kB TF; RelB.

FIG. 1G shows a heatmap with Ingenuity pathway analysis (IPA) of LPS associated DEGs. IPA of the LPS-stimulated DEGs showed a severe loss in the ability of p50^{-KO}, p52^{-KO}, p65^{-KO} and c-Rel^{-KO} cells to induce activation of inflammatory pathways including TLR, TNFR2, and IL-6 signaling, in response to LPS. In RelB^{-KO} cells, though, the inflammatory pathways were only moderately impacted and responded almost in a similar manner to that seen in the CTR cells. Overall, this further shows that the canonical NF-kB TFs are the major driver of LPS induced inflammatory response compared to non-canonical NF-kB TF; RelB. The colour gradient in the heatmap represents the significant changes (−log 10 p) in the expression of pathways associated genes. The −log 10 p>1.33 considered significant and Z score represents the directionality of the pathways, + indicates increased activity, − decreased activity and no symbols indicate lack of prediction by the IPA.

FIG. 1H shows a heatmap with Spearman's ranked correlation between independent clones of factors specific NF-kB KOs based on the total set of DEGs in unstimulated cells. The spearman's correlation analysis based on 1258 DEGs showed homogeneity among independent KO clones as their correlations were highest among all the KO clones. Also, correlation analysis captured a known functional interaction between RelB and p65^{-KO} clones as their independent KO clones clustered together.

FIG. 1I shows a heatmap with IPA analysis of unstimulated DEGs. IPA analysis of the unstimulated DEGs in different NF-kB KO cells showed an enrichment of metabolic and cancer pathways, which were overrepresented in the p65^{-KO} cells. Taken together, global gene expression analysis of NF-kB KO human AML cell line U937 suggests state-specific homo- and heterogeneity among transcriptional regulation by NF-κB family of TFs. The colour gradient in the heatmap represents the significant changes (−log 10 p value) in the expression of pathways associated genes. The −log 10 p value >1.33 considered significant and Z score represents the directionality of the pathways, + indicates increased activity, − decreased activity and no symbols indicate lack of prediction by the IPA.

FIG. 2A shows a heatmap with the dentification of p65 homodimer regulated target genes in DEGs from unstimulated NFkB KOs vs CTR. DEGs were merged with the ChIP-ChIP binding profile to identify 595 overlapping genes. Modular analysis resulted in identification of p65-p65 homodimer specific modules (downregulated modules including p65 are indicated by light grey boxes with-symbol and upregulated modules are indicated by dark grey boxes with + symbol) encompassing 225 genes.

FIG. 2B enrichment analysis for disease functions within p65-p65 homodimer specific 225 DEGs by IPA analysis. The metabolic and cancer associated pathways remained to be significantly enriched in IPA analysis of p65 homodimer specific DEGs (225 genes) further strengthened the potential role for the p65 homodimer in regulating important aspects of metabolism and cancer. The colour gradient in the heatmap represents the significant changes (−log 10 p value) in the expression of pathways associated genes. The −log 10 p>1.33 considered significant and Z score represents the directionality of the pathways, + indicates increased activity, − decreased activity and no symbols indicate lack of prediction by the IPA.

FIG. 2C shows a heatmap with the expression of genes associated with metabolic pathways from IPA across NF-kB KOs and CTR cells. The p65 deficiency induced deregulation of metabolic pathways such as OXPHOS, sirtuin signaling pathway and mitochondrial dysfunction in AML cells was further highlighted by the distinct regulation of the genes involved in these pathways in p65^{-KO} cells as compared with CTR and the other NF-κB KO cells. This further implies p65-mediated regulation of the metabolic pathways in U937 AML cells.

FIG. 2D shows genome browser shot and plots with the binding pattern of NF-kB TFs across the locus of AKT1 and COX6A1 genes (lower panel) and the respective RNA-seq reads in KOs and CTR cells (above panel).

FIG. 2E shows plots of mitostress analysis of mitochondrial functions using oxygen consumption rate (OCR) for OXPHOS. Combine data of N=4 clones for both p65 KOs and CTR is shown.

FIG. 2F shows graphs with individual OXPHOS parameter data from FIG. 2E

FIG. 2G shows plots of glycolysis stress analysis of extracellular acidification rate (ECAR) for lactate production for four clones of p65^{-/-} KOs and CTR.

FIG. 2H shows graphs with individual glycolysis parameter data from FIG. 2G.

FIG. 2I shows a plot with cell energy phenotype analysis using OCR and ECAR measurements in Mito stress test, demonstrating high “hybrid” energetic state in p65^{-KO} clones. Data from F and His Mean±SD, *, P<0.05, Mann Whitney test.

FIG. 3A shows a heatmap with mRNA expression of p65 homodimer regulated genes associated with cancer pathway across NF-kB TFs KO and CTR. The p65 deficiency induced deregulation of cancer pathways including AML and AML-associated signaling pathways such as IL-6, IL-7, CXCR4, JAK-STAT and GM-CSF signalling in AML cells was highlighted by the distinct regulation of the genes involved in these pathways in p65^{-KO} cells as compared with CTR and the other NF-κB KO cells. This further suggests a potential inhibitory role of homodimeric p65 in regulating leukemia-specific pathways.

FIG. 3B shows images (left panel) and graphs (right panel) with CFU analysis of p65-KO and CTR clones (N=5 clones) and compiled data showing frequency of total colonies formed with 500 cells.

FIG. 3C show a schematic representation of induced AML xenografts in huNOG mice.

FIG. 3D and FIG. 3E show graphs with weight loss and Kaplan-Meier survival curve, respectively, of huNOG mice receiving either p65^{-KO} or CTR U937 cells (N=15-17 mice in each group, data of two experiments).

FIG. 3F show flow cytometry dot plots with blood and tissue (BM and spleen) analysis of CTR or p65^{-KO} induced xenograft expansion in huNOG mice. hCD45⁺GFP⁺ AML cells have been indicated by box.

FIG. 3G shows graphs with compiled data of blood flow cytometry analysis of CTR or p65^{-KO} induced xenograft expansion in huNOG mice at average 8- and 13-days post injection in two different experiments.

FIG. 3H shows graphs with BM and spleen flow cytometry analysis of CTR or p65-KO induced xenograft expansion in huNOG.

FIG. 3I and FIG. 3J show plots with correlation analysis of xenografts expansion (GFP+ AML cells in blood) and humanization (i.e., chimerism in blood) before CTR or p65-KO xenograft induction with survival in huNOG mice.

FIG. 3K show graphs with comparison of total blood human CD45⁺ cells in controls and p65^{-KO} xenografts in huNOG mice.

FIG. 3L show plots with correlation analysis of xenografts expansion (hCD45⁺GFP⁺ cells) and normal haematopoiesis (hCD45⁺GFP⁻ cells) in blood in huNOG mice injected with CTR or p65-KO AML cells.

FIG. 4A shows a schematic depiction of using 441 p65 specific DEGs to analyse AML patient data (Cox regression results for the datasets TARGET and TCGA, left panel), topmost genes from meta-analysis of the p65/p65 homodimer regulated gene are indicated in a tabular format (right panel).

FIG. 4B shows a forest plot displaying hazard ratios and 95% CIs (confident interval) of ATP13A2 from univariate Cox regression analysis in TARGET and TCGA datasets. Meta HR (hazard ratio) and CI is indicated.

FIG. 4C shows Kaplan-Meier curves for ATP13A2 as gene predictor to stratify survival in AML patients.

FIG. 4D show images and graphs with CFU analysis of p65^{-KO} (N=5 clones), CTR (N=5 clones) and p65^{-KO}-shATP13A2 (n=3) clones (left panel) and compiled data showing frequency of total colonies formed with 500 cells (right panel).

FIG. 4E shows schematic representation of p65-KO, CTR and p65^{-KO}-shATP13A2 induced AML xenografts experiments in huNOG mice.

FIG. 4F shows graphs with blood and tissue (BM and spleen) flow cytometry analysis of p65^{-KO}, CTR and p65^{-KO}-shATP13A2 induced xenograft expansion in huNOG mice.

FIG. 4G shows Kaplan-Meier survival curve of huNOG mice receiving either p65^{-KO}, CTR and p65^{-KO}-shATP13A2 (N=5 mice in each group).

FIG. 4H shows H & E staining of BM of p65^{-KO}, CTR and p65^{-KO}-shATP13A2 induced xenografts in huNOG mice. Magnification—20× and 60×. Normal bone marrow (placebo group left panel) shows the presence of myeloid series (Ms), Erythroid series (Es) and megakaryocytes (Mg), Sinusoids(S) interlaces within the hematopoietic cells. Infiltrated AML cells indicated by black arrow. Right panel showing overall pathological score.

FIG. 4I shows H & E staining of spleen from huNOG mice which received p65^{-KO}, CTR and p65^{-KO}-shATP13A2 induced xenografts. Magnification—20× and 100×. Images indicate normal spleen (placebo group left panel), which contains white pulp (WP), Red pulp (RP) and EMH (left panels). Infiltrated AML cells indicated by star and apoptosis (by black arrow) are shown in spleen from xenograft carrying huNOG mice. Right panel showing overall pathological score.

FIG. 5A shows gene signature enrichment analysis (GSEA) on p65^{-KO} specific 441 DEGs, demonstrating enrichment of KEGG_Lysosome pathway. The expression of selected genes in Control and p65^{-KO} cells is shown at the bottom as a heatmap. The heatmap shows the expression of respective genes among three separate p65-KO and CTRL clones.

FIG. 5B shows histogram overlay plot showing the mean fluorescence intensity (MFI) of pTFEB (left panel) and TFEB (right panel) proteins in p65^{-KO} p65^{-KO}-shATP13A2 and CTR cells, assessed by flowcytometry. The respective compiled data from 3 clones is shown at the bottom as bar graph.

FIG. 5C shows an immunoblot showing protein expression of pTFEB, TFEB and GAPDH in control, p65^{-KO}, p65^{-KO}-shATP13A2 and CTR cells.

FIG. 5D shows histogram overlay plot with the mean fluorescence intensity (MFI) of intracellular LAMP1 (left panel). The bar diagram (right panel) represents the MFI for three clones from each group in p65^{-KO}, p65^{-KO}-shATP13A2 and CTR cells as measured by flow cytometry.

FIG. 5E shows LAMP1 analysis by immunofluorescence staining and confocal microscopy in p65^{-KO}, p65^{-KO}-shATP13A2 and CTR cells (representative images, left panel). Right panel shows the compiled data of MFI per cell analysed by ImageJ software (100 cells per group).

FIG. 5F shows histogram overlay plots (left panel) of MFI for lysotracker in p65^{-KO}, p65^{-KO}-shATP13A2 and CTR cells as measured by flowcytometry. Right panel shows the compiled data of MFI in bar graph.

FIG. 5G shows a graph with mito stress analysis of mitochondrial functions using oxygen consumption rate (OCR) for OXPHOS, measured using Seahorse analyzer. Combine data of N=3 clones for all three groups p65^{-KO}, p65^{-KO}-shATP13A2 and CTR is shown.

FIG. 5H shows a graph with glycolysis stress analysis of extracellular acidification rate (ECAR), measured using Seahorse analyzer, for lactate production among all three cell types.

FIG. 5I shows a graph with individual glycolysis parameter data from FIG. 5H.

FIG. 5J shows a graph with cell energy phenotype analysis using OCR and ECAR measurements in Mito stress test data from 5G and 5I, demonstrating high “hybrid” energetic state in p65-KO clones.

FIG. 5K shows schematic representation (left panel) of CTR and shATP13A2 induced AML xenografts experiments in huNOG mice. Right panel is Kaplan-Meier survival curve of huNOG mice receiving control or shATP13A2 cells (N=5-6 mice in each group).

FIG. 6A shows a heat map representing the levels of human cytokine and chemokine in the serum of xenografts of control and p65-KO U937 cells (samples from huNOG mice in FIG. 3E). Significantly modulated cytokines/chemokines across control and p65^{-KO} xenograft groups (n=6 mice in each group) are indicated by *, Student t-test. Analysis of human cytokines and chemokines was performed using Luminex assay in the serum derived from huNOG xenografts. Heatmap showed several cytokines/chemokines such as MDC (CCL22), IL-10, IL-5, IP-10 and PDGF-AA to be significantly modulated in the serum of p65-KO xenografts compared to huNOG mice which received control xenografts. MDC and IL5 were increased, and IL-10, IP-10 and PDGF-AA were decreased. Taken together this data suggest an anti-leukemic role of p65 where its reduction has an impact on normal haematopoiesis through competitive advantage of p65-KO AML cells and also induces pro-leukemic cytokine signature in xenografts.

FIG. 6B shows a bar graph of the key modulated cytokines in p65^{-KO} induced xenografts compared to controls in FIG. 6A. *, P<0.05; **, P<0.01, Student t-test.

FIG. 7A shows histological images with the analysis of pathological changes at 20×, 60× through H&E staining of BM from huNOG mice which were injected with either control AML cells (left panel, n=5), p65^{-KO} AML cells (middle panel, n=5) or p65^{-KO}-ATP13A2-KD AML cells (right panel, n=4). Necrosis (N) as evidenced with the presence of nuclear pyknosis (arrow) have been depicted. Affected area becomes hypo cellular and is replaced with amorphous granular eosinophilic materials (#) and is often accompanied with haemorrhages (*). The Right graph shows overall pathological score based on subjective scoring for necrosis and haemorrhages.

FIG. 7B show histological images of Induced pathological changes in the spleen (20×) of mice in FIG. 7B showing lymphoid depletion (see indicated area by empty black box) in p65-KO xenografts compared to intact lymphoid region in Control xenograft (injected with CTR AML cells;) or in p65-KO-shATP13A2 xenograft (injected with p65-KO-shATP13A2 AML cells). White pulp area (WP) is marked by black box, infiltrated AML cells are indicated by arrow.

FIG. 7C shows a heat map representing the normalized levels of human cytokine and chemokine in the plasma of mice xenografts in A. Significantly modulated cytokines/chemokines across control (n=9); p65^{-KO} (n=8) and p65^{-KO}-ATP13A2-KD (n=8) xenograft groups are indicated by *, One way ANOVA. Analysis of human cytokines/chemokines showed a reduced inflammatory state in huNOG mice which received ATP13A2-depleted p65KO AML cells. Importantly, trend of CCL22, IL10, IL1RA, PDGF-AA, FLT3-G and VEGF; analytes that are known to be associated with AML pathologies and poor survival in AML patient, was reversed in the serum of huNOG mice receiving ATP13A2-depleted p65KO AML cells. This reversal corroborated with the reversal in lymphoid depletion (FIG. 7B). Taken together, our data links the aggressive nature of p65-KO cells with pro-leukemic cytokine and chemokine signature and immune suppression driven through ATP13A2.

FIG. 7D shows bar graphs showing the significantly reversed p65-KO associated cytokines/chemokines in p65^{-KO}-ATP13A2-KD xenografts. P value, one way ANOVA.

FIG. 8A shows a schematic representation of targeted exons of NF-kB TFs genes by pgRNA CRISPR-Cas9 approach. First five exons of NFKB1, NFKB2, RELA, REL and RELB genes are indicated.

FIG. 8B shows sequencing plots with the validation of editing at targeted regions through PCR amplification using gene specific primers and Sanger sequencing.

FIG. 8C shows immuno blots with the validation for the loss of protein in the identified edited clones of NF-kB TFs by western blotting. Respective clones identified, validated and selected for further experiments are marked by letters in the rectangle box.

FIG. 9 shows a workflow used for analysing RNA-seq data.

FIG. 10 shows volcano plots with differential gene expression in LPS stimulated and unstimulated KOs. Volcano plots showing significance versus fold changes of gene in differential gene expression analysis among various clones. Both untreated (UT) and LPS-treated conditions are shown. Left and right-side genes of the dashed lines (depicted as dots) represent respectively up and down regulated genes selected based on P value <0.005 and log₂ fold change >1.5.

FIG. 11 shows genome browser shot and plots with the binding pattern of NF-kB TFs across the locus of IL-6R and PLCG2 genes (lower panel) and the respective RNA-seq reads in those loci for KOs and CTR cells (above panel).

FIG. 12A shows a schematic representation of the generation of U937 induced AML xenografts in NOG mice.

FIG. 12B and FIG. 12C show plots with weight loss and Kaplan-Meier survival curve of NOG mice receiving either p65^{-KO} or CTR U937 cells (N=7-8 mice in each group, data of two experiments).

FIG. 12D shows dot plots with flow cytometry gating strategy and analysis of xenografts expansion in the blood of NOG recipient mice at 11-16 days' post injection and Blood flow cytometry analysis of CTR or p65^{-KO} induced xenograft expansion in NOG mice at 11-16 days' post injection. hCD45⁺GFP⁺ AML cells have been indicated in green.

FIG. 12E shows Kaplan-Meier survival curve of NOG mice receiving either p50^{-KO}, p52^{-KO}, c-Rel^{-KO}, RelB^{-KO} or CTR U937 cells (N=4-5 mice in each group except RelB). Survival curve of RelB and CTR groups is based on three experiments (N=13-14 mice in each group).

FIG. 13A shows a schematic representation of the generation of U937 induced AML xenograft model in huNOG mice.

FIG. 13B shows flow cytometry gating strategy and analysis of xenografts expansion in the blood, BM and spleen of huNOG recipient mice at 11-16 days' post injection.

FIG. 14A shows flow cytometry gating strategy and analysis of xenografts expansion in the blood of huNOG recipient mice at 11-16 days' post injection. Analysis of cellular phenotypes (expression of hCD123, hCD117, hCD244 and hCD45RA) of expanded xenografts (hCD45⁺GFP⁺ cells) in blood is indicated by histogram overlay plots.

FIG. 14B shows data analysed as in FIG. 14A for cellular phenotypes of expanded xenografts in blood, indicated by histogram overlay plots. Respective compiled data of specific phenotype marker in different huNOG mice is shown as bar graph at bottom.

FIG. 15 shows survival plots with signature of 3 and 4 gene combinations of p65/p65 homodimer regulated gene that improves prediction of overall survival in TCGA and TARGET cohorts. Gene combination is depicted at the top of each plot.

FIG. 16A shows a schematic representation of shRNA knockdown in p65-KO cells showing shRNA sequences, cloning, infection and selection.

FIG. 16B shows bar graphs with qPCR validation of ATP13A2 knockdown in p65-KO cells.

FIG. 17 shows a graph with individual OXPHOS parameter data from FIG. 5G.

FIG. 18A shows pAdaptor and pDonor plasmids used to generate the pgRNA.

FIG. 18B shows gateway compatible CRISPR/CAS9 expression plasmid; CAS9-T2AGFP for knock out generation.

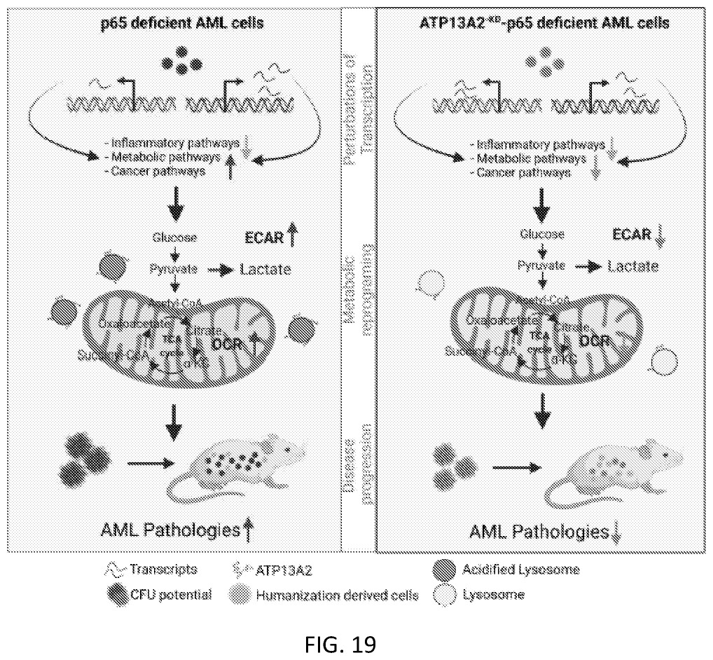

FIG. 19 depicts a schematic representation of proposed model showing a novel p65-ATP13A2 mediated control of AML pathogenesis: Left box represents the sequence of events that occur when p65 is depleted in AML cell line U937, which in turn leads to increased expression of ATP13A2, a lysosomal transporter. p65-deficient AML cells shows deregulation of energetic and lysosomal pathways, leading to enhanced AML pathologies in xenografts humanized mice model. Right box represents the sequence of events when ATP13A2 expression level is reduced in p65-deficient AML cells, leading to the reversal of p65-mediated metabolic reprograming and pathology in AML cells in humanized mice. Overall, the inventors of the present disclosure propose that p65 deficiency exacerbates AML pathologies via a ATP13A2-mediated mechanism, which involves metabolic adaptation and lysosomal dysfunction.

### Applications

Embodiments of the methods disclosed herein provide a method of identifying a therapeutic target for treating myeloid leukemia (AML) using a genetically modified cell and a method of treating acute myeloid leukemia (AML) in a subject in need thereof.

Advantageously, the present invention identifies ATP13A2 (also known as Park9) as a therapeutic target for AML.

Even more advantageously, the present invention provides the development of clinical candidates against ATP13A2 for the treatment of AML.

Even more advantageously, the present invention provides the development and use of ATP13A2 as a target for the treatment of AML.

Even more advantageously, the present invention provides a targeted therapy for AML and development of combinatorial therapies (in conjunction with standard chemotherapy) for AML.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A genetically modified cell wherein at least one gene has been deleted from the cell and the gene is selected from the group consisting of p65 (RELA), p50 (NFKB1), p52 (NFKB2), c-Rel (REL), and RelB (RELB).

2. A method of identifying a therapeutic target for treating acute myeloid leukemia (AML) in a subject, the method comprising
providing a genetically modified cell wherein at least one gene has been deleted from the cell and the gene is selected from the group consisting of p65 (RELA), p50 (NFKB1), p52 (NFKB2), c-Rel (REL), and RelB (RELB).

3. A method of treating acute myeloid leukemia (AML) in a subject in need thereof, comprising modulating the activity of an NF-KB pathway.

4. The method according to claim 3, wherein the modulating of an NF-KB pathway comprises modulating multiple pathways comprising a metabolic pathway, an inflammatory pathway, a cancer associated pathway, and combinations thereof.

5. The method according to claim 3, wherein the NF-KB pathway gene is p65.

6. The method of claim 3, wherein the modulating the activity of NF-KB pathway comprises modulating a metabolic pathway comprising oxidative phosphorylation (OXPHOS), mitochondrial dysfunction, Sirtuin signaling and/or mTOR signaling.

7. The method of claim 3, wherein the modulating the activity of NF-KB pathway comprises modulating the cancer associated pathway comprises pathways related to acute myeloid leukemia (AML), small lung cancer, and/or pancreatic adenocarcinoma, optionally, wherein the cancer associated pathway comprises AML and AML-associated signaling pathways comprising IL-6, IL-7, JAK, CXCR4, JAK-STAT and/or GM-CSF.

8. The method of claim 3, wherein the modulating of NF-KB pathway comprises modulating the inflammatory pathway comprises TLR, TNFR2, CCL22, IL-10, IL-17A, CD40 and/or IL-6 signalling.

9. The method of claim 3, wherein the modulating the activity of the NF-kB comprises regulating the gene and/or protein expression of a lysosomal-associated protein.

10. The method of claim 3, wherein the modulating the activity of the NF-KB comprises regulating the function of a lysosomal-associated protein.

11. The method of claim 10, wherein the lysosomal-associated protein is ATP13A2/Park9.

12. The method of claim 3, wherein the method comprises administering an agent that inhibits the activity of the ATP13A2 gene.

13. The method of claim 3, wherein the method of treating reduces one or more indications comprising reduction of oxygen consumption rate (OCR), reduction in maximum respiration, reduction in spare respiratory capacity, reduction of nonmitochondrial respiration, and combinations thereof.

14. The method of claim 3, wherein the method of treating reduces one or more indications comprising reduction in glycolysis, reduction in glycolytic capacity, reduction in maximum mitochondrial respiration, and combinations thereof.

15. The method of claim 3, wherein the method of treating reverses or improves dysfunctional mitochondrial function.

16. The method of claim 3, wherein the method of treating reduces one or more indications comprising elimination of ability to proliferate and/or form colonies.

17. The method of claim 3, wherein the method further comprising treating with inhibiting agents comprising a chemotherapeutic agent, an immune therapy agent, a cellular therapy agent, an oligonucleotide, an antigen binding molecule, a small molecule inhibitor, and/or combinations thereof.

18. The method of claim 17, wherein the method further comprising treating with a chemotherapeutic agent.

19.-21. (canceled)


SEQUENCE LISTING (45 sequences)

SEQ ID NO: 1 - DNA, 20 bp, synthetic construct
  source 1..20: mol_type = other DNA
tgttcaggccttcccaaata

SEQ ID NO: 2 - DNA, 20 bp, synthetic construct
  source 1..20: mol_type = other DNA
tatcaccatggaattcatgc

SEQ ID NO: 3 - DNA, 20 bp, synthetic construct
  source 1..20: mol_type = other DNA
caggtagtccaccatgggat

SEQ ID NO: 4 - DNA, 20 bp, synthetic construct
  source 1..20: mol_type = other DNA
gaacaagaagtcttaccctc

SEQ ID NO: 5 - DNA, 20 bp, synthetic construct
  source 1..20: mol_type = other DNA
gtagcaactctccatgtctc

SEQ ID NO: 6 - DNA, 20 bp, synthetic construct
  source 1..20: mol_type = other DNA
gggcaggcggcatgactcac

SEQ ID NO: 7 - DNA, 20 bp, synthetic construct
  source 1..20: mol_type = other DNA
caataataccatccagaccc

SEQ ID NO: 8 - DNA, 20 bp, synthetic construct
  source 1..20: mol_type = other DNA
tgaacttgctgacctgtttc

SEQ ID NO: 9 - DNA, 20 bp, synthetic construct
  source 1..20: mol_type = other DNA
gaccccggccatggacggtg

SEQ ID NO: 10 - DNA, 20 bp, synthetic construct
  source 1..20: mol_type = other DNA
cgcgggcccagctgcgaccc

SEQ ID NO: 11 - DNA, 20 bp, synthetic construct
  source 1..20: mol_type = other DNA
ttccccctcatcttcccggc

SEQ ID NO: 12 - DNA, 20 bp, synthetic construct
  source 1..20: mol_type = other DNA
tccttgtgagaatgggcatt

SEQ ID NO: 13 - DNA, 20 bp, synthetic construct
  source 1..20: mol_type = other DNA
cccgcgtgcatgcttcggtc

SEQ ID NO: 14 - DNA, 20 bp, synthetic construct
  source 1..20: mol_type = other DNA
ttacctaaggcccccagctc

SEQ ID NO: 15 - DNA, 20 bp, synthetic construct
  source 1..20: mol_type = other DNA
gtactcgtcgatgatctctg

SEQ ID NO: 16 - DNA, 20 bp, synthetic construct
  source 1..20: mol_type = other DNA
gccacgcctggtgtctcgcg

SEQ ID NO: 17 - DNA, 20 bp, synthetic construct
  source 1..20: mol_type = other DNA
tgaacactcaccggaggcca

SEQ ID NO: 18 - DNA, 20 bp, synthetic construct
  source 1..20: mol_type = other DNA
cgcgccccatgaacactcac

SEQ ID NO: 19 - DNA, 20 bp, synthetic construct
  source 1..20: mol_type = other DNA
atcagcaggcagcattccag

SEQ ID NO: 20 - DNA, 20 bp, synthetic construct
  source 1..20: mol_type = other DNA
ggtctattacctggatagaa

SEQ ID NO: 21 - DNA, 19 bp, synthetic construct
  source 1..19: mol_type = other DNA
tggcgagcttaacacgagg

SEQ ID NO: 22 - DNA, 23 bp, synthetic construct
  source 1..23: mol_type = other DNA
aacaatatgtgtgaaagcgggtg

SEQ ID NO: 23 - DNA, 23 bp, synthetic construct
  source 1..23: mol_type = other DNA
tggcagcagcaatttaagacaag

SEQ ID NO: 24 - DNA, 23 bp, synthetic construct
  source 1..23: mol_type = other DNA
gggtactttcaggctctctatgg

SEQ ID NO: 25 - DNA, 20 bp, synthetic construct
  source 1..20: mol_type = other DNA
atctcgctctccaccggatc

SEQ ID NO: 26 - DNA, 22 bp, synthetic construct
  source 1..22: mol_type = other DNA
cacctcccatctccaagtcttc

SEQ ID NO: 27 - DNA, 20 bp, synthetic construct
  source 1..20: mol_type = other DNA
gggaggatgctgagtcaagg

SEQ ID NO: 28 - DNA, 20 bp, synthetic construct
  source 1..20: mol_type = other DNA
aagtgagtaatcggcggacc

SEQ ID NO: 29 - DNA, 21 bp, synthetic construct
  source 1..21: mol_type = other DNA
tgacccggggtgcaagaattc

SEQ ID NO: 30 - DNA, 25 bp, synthetic construct
  source 1..25: mol_type = other DNA
ttcgtcctcctttggttcgcttatg

SEQ ID NO: 31 - DNA, 19 bp, synthetic construct
  source 1..19: mol_type = other DNA
gcctctcccagccaatctc

SEQ ID NO: 32 - DNA, 23 bp, synthetic construct
  source 1..23: mol_type = other DNA
tgtggagatgactgtgaagaaac

SEQ ID NO: 33 - DNA, 21 bp, synthetic construct
  source 1..21: mol_type = other DNA
ctcagttgtctcgtccagagc

SEQ ID NO: 34 - DNA, 22 bp, synthetic construct
  source 1..22: mol_type = other DNA
ctcatcaccctctgccttcttc

SEQ ID NO: 35 - DNA, 24 bp, synthetic construct
  source 1..24: mol_type = other DNA
tagctgggattacagacgtgggag

SEQ ID NO: 36 - DNA, 19 bp, synthetic construct
  source 1..19: mol_type = other DNA
cactcgtagcggaagcgca

SEQ ID NO: 37 - DNA, 20 bp, synthetic construct
  source 1..20: mol_type = other DNA
tgtaaaacgacggccagtgc

SEQ ID NO: 38 - DNA, 23 bp, synthetic construct
  source 1..23: mol_type = other DNA
gctatgaccatgattacgccacg

SEQ ID NO: 39 - DNA, 22 bp, synthetic construct
  source 1..22: mol_type = other DNA
gttatccaggctctgcgaagga

SEQ ID NO: 40 - DNA, 22 bp, synthetic construct
  source 1..22: mol_type = other DNA
gtggacgatgatcagatgctcc

SEQ ID NO: 41 - DNA, 21 bp, synthetic construct
  source 1..21: mol_type = other DNA
gaggcactgaacccctactat

SEQ ID NO: 42 - DNA, 21 bp, synthetic construct
  source 1..21: mol_type = other DNA
gcaccatctacagcatcttca

SEQ ID NO: 43 - DNA, 21 bp, synthetic construct
  source 1..21: mol_type = other DNA
gtgcctctgaatgagattgta

SEQ ID NO: 44 - DNA, 21 bp, synthetic construct
  source 1..21: mol_type = other DNA
gcgcgatagcgctaataattt

SEQ ID NO: 45 - DNA, 21 bp, synthetic construct
  source 1..21: mol_type = other DNA
cgctgagtacttcgaaatgtc